(19) United States
(12) Patent Application Publication
(10) Pub. No.: US 2024/0216692 A1
(43) Pub. Date: Jul. 4, 2024

(54) STIMULATING A GLOSSOPHARYNGEAL-RELATED TISSUE FOR UPPER AIRWAY PATENCY

(71) Applicant: INSPIRE MEDICAL SYSTEMS, INC., Golden Valley, MN (US)

(72) Inventors: Wondimeneh Tesfayesus, New Brighton, MN (US); Kevin Verzal, Lino Lakes, MN (US)

(73) Assignee: INSPIRE MEDICAL SYSTEMS, INC., Golden Valley, MN (US)

(21) Appl. No.: 18/394,963

(22) Filed: Dec. 22, 2023

Related U.S. Application Data
(60) Provisional application No. 63/477,702, filed on Dec. 29, 2022.

(51) Int. Cl. A61N 1/36

(52) U.S. Cl. CPC A61N 1/3611; A61N 1/36057

(57) ABSTRACT

Examples are directed to an apparatus, device and/or method comprising selectively stimulating, via at least one stimulation element, glossopharyngeal-related tissue of a patient to promote upper airway patency.

### CROSS-REFERENCE TO RELATED APPLICATIONS

This application claims the benefit of the filing date of U.S. Provisional Application Ser. No. 63/477,702, filed Dec. 29, 2022 and entitled “Stimulating a Glossopharyngeal-Related Tissue for Upper Airway Patency,” the entire teachings of which are incorporated herein by reference.

### BACKGROUND

An obstructed upper airway may cause significant health problems and is common among the adult population. Upper airway obstructions can arise from a variety of factors, conditions, etc. Among other consequences, for some patients such obstructions may result in sleep disordered breathing such as (but not limited to) obstructive sleep apnea. Some forms of treatment of sleep disordered breathing may include electrical stimulation of nerves and/or muscles relating to upper airway patency.

### BRIEF DESCRIPTION OF THE DRAWINGS

FIG. 1 is a flow diagram schematically representing an example method comprising stimulating a glossopharyngeal-related tissue.

FIGS. 2A-2D illustrate an example upper airway of a patient and at least one stimulation element for stimulating a glossopharyngeal-related tissue.

FIGS. 3A-3D illustrate example target locations for stimulation of the glossopharyngeal-related tissue and surrounding anatomy.

FIGS. 4A-4B illustrate example timing diagrams for a carbon dioxide sensor of the body.

FIGS. 5A-5F are diagrams schematically representing deployment of example stimulation elements and example implantable medical devices.

FIG. 6A is a block diagram schematically representing an example control portion.

FIG. 6B is a diagram schematically illustrating at least some example arrangements of a control portion.

FIG. 7 is a block diagram schematically representing a user interface.

FIG. 8 is a block diagram which schematically represents some example implementations by which an implantable device may communicate wirelessly with external circuitry outside the patient.

FIGS. 9A-9B are diagrams schematically representing an example method comprising selectively applying different care and a control portion.

FIG. 9C is a block diagram schematically representing an example sensing portion.

FIG. 10 is a block diagram schematically representing a stimulation portion of a care engine.

FIG. 11 is a diagram schematically representing a patient's body, implantable components, and/or external elements of an example device and/or for use in an example method.

FIGS. 12A-12D are diagrams including front and side views schematically representing patient anatomy and example methods relating to collapse patterns associated with upper airway patency.

FIGS. 12E-12H are block diagrams schematically representing example devices and/or example methods relating to collapse patterns associated with upper airway patency.

FIGS. 13 and 14 each are a block diagram schematically representing an example care engine and control portion, respectively.

FIG. 15 is a block diagram schematically representing an example user interface.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

At least some examples of the present disclosure are directed to example apparatuses and/or devices for, and/or example methods of therapy and/or other care of medical conditions which may relate to upper airway patency. At least some of the examples comprise use of a medical device in order to increase or maintain upper airway patency. At least some of the examples of the present disclosure may be employed to treat sleep disordered breathing (SDB), which may comprise obstructive sleep apnea (OSA) and/or other types of SDB.

SDB may be treated using a variety of different techniques. In some instances, external breathing therapy devices, such as a continuous positive airway pressure (CPAP) machine or other devices which provide air pressure to the patient during sleep, are used to treat patients. Such external breathing devices may not work for all patients and may be bothersome to the patients, resulting in reduced use. Such patients may sometimes be referred to as being non-compliant or non-adherent because they fail to comply with the prescribed therapy.

For some patients and types of SDB, surgical interventions may be used to improve symptoms, such as uvulopalatopharyngoplasty, lateral pharyngoplasty, lingual tonsillectomy, and tongue reduction surgery, among other procedures. However, such surgical interventions have a mixed record of success for many cases of SDB.

On the other hand, for most patients exhibiting moderate and severe obstructive sleep apnea, great success has been found with the use of some types of implantable neurological devices that provide electrical stimulation to the hypoglossal nerve, which causes the tongue muscle to stiffen and the tongue to protrude, thereby promoting upper airway patency (e.g., dilating the upper airway). Nevertheless, a small percentage of patients may not respond to such treatments and therefore may sometimes be referred to as “non-responders”. The anatomical structures involved in upper airway patency may sometimes herein be referred to as upper airway patency-related tissue, which may comprise nerves, and/or muscle, which when stimulated may maintain, restore, and/or increase upper airway patency. Further details regarding such anatomical structures are provided below.

As least some examples of the present disclosure are directed to methods, apparatuses, and/or devices comprising stimulating, via at least one stimulation element, at least one fiber of a glossopharyngeal nerve of a patient and which may be used to treat a SDB patient. In some such examples, the SDB patients are non-responders to, and/or which are non-compliant with, other types of SDB treatments. In some such examples, the stimulation may be selectively applied. Examples are not so limited and may include stimulating other types of glossopharyngeal-related tissue.

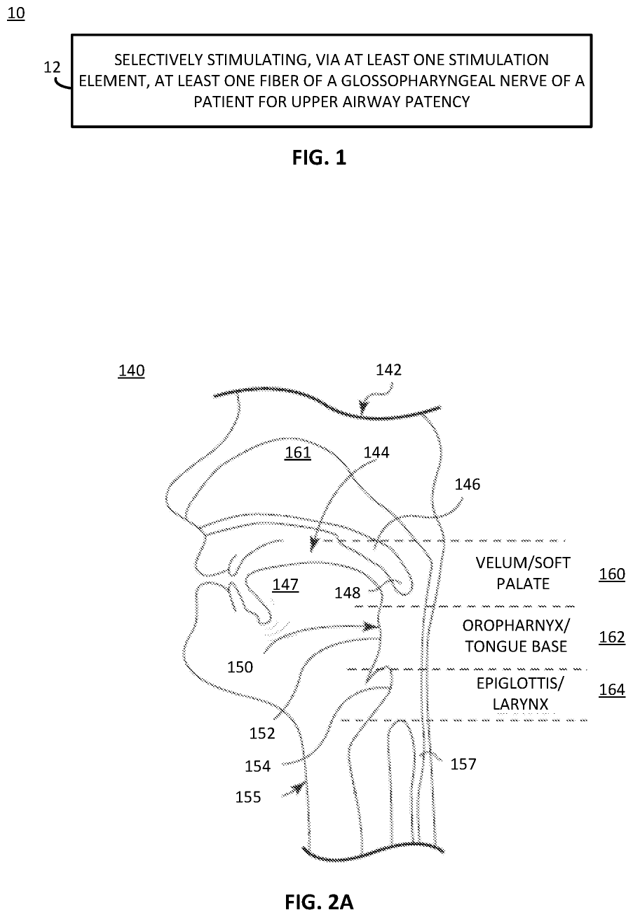

These examples, and additional examples, are described in connection with at least FIGS. 1-15.

FIG. 1 is a flow diagram schematically representing an example method comprising stimulating a glossopharyngeal-related tissue for upper airway patency. In some examples, the stimulation may be applied selectively, and in some examples, the glossopharyngeal-related tissue may comprise a glossopharyngeal nerve. Accordingly, in some examples the method 10 may comprise selectively stimulating, via at least one stimulation element, at least one fiber of a glossopharyngeal nerve of a patient to promote upper airway patency, as shown at 12 in FIG. 1. In some examples, selective stimulation of the least one fiber of the glossopharyngeal nerve causes an increase of upper airway patency and/or maintains the upper airway patency. In some examples, other types of glossopharyngeal-related tissue may be stimulated, such as at least one muscle innervated by the glossopharyngeal nerve, as further described herein. In some examples, glossopharyngeal-related tissue may comprise at least one stylopharyngeus muscle and/or at least one fiber of a glossopharyngeal nerve.

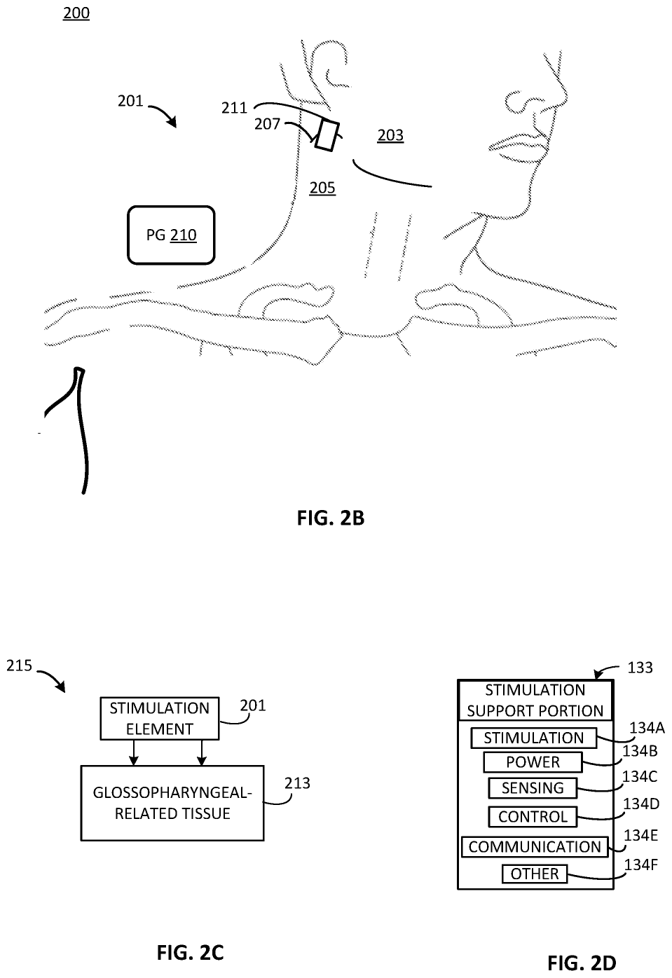

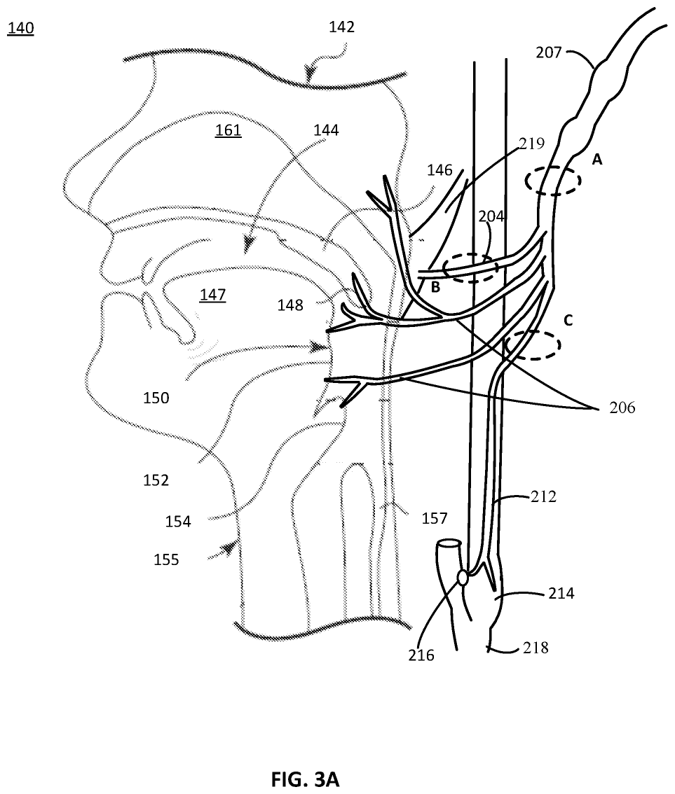

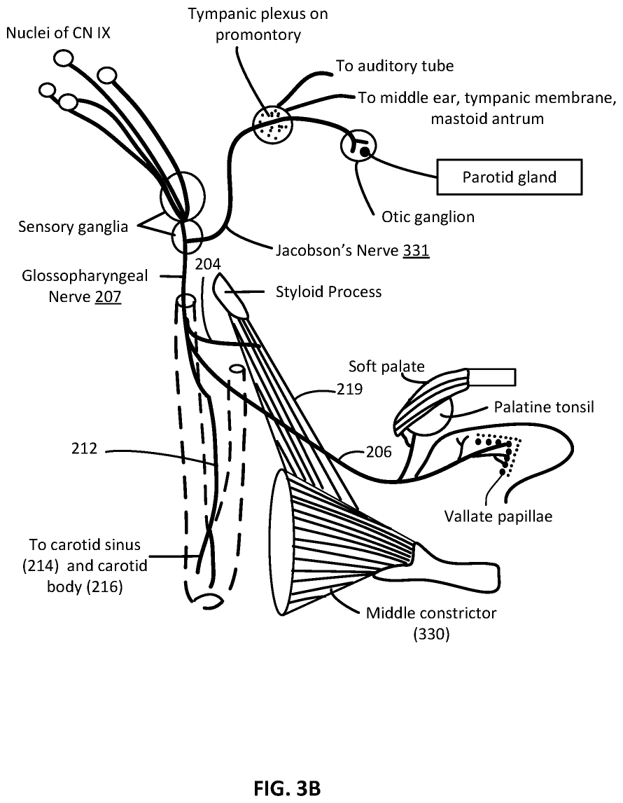

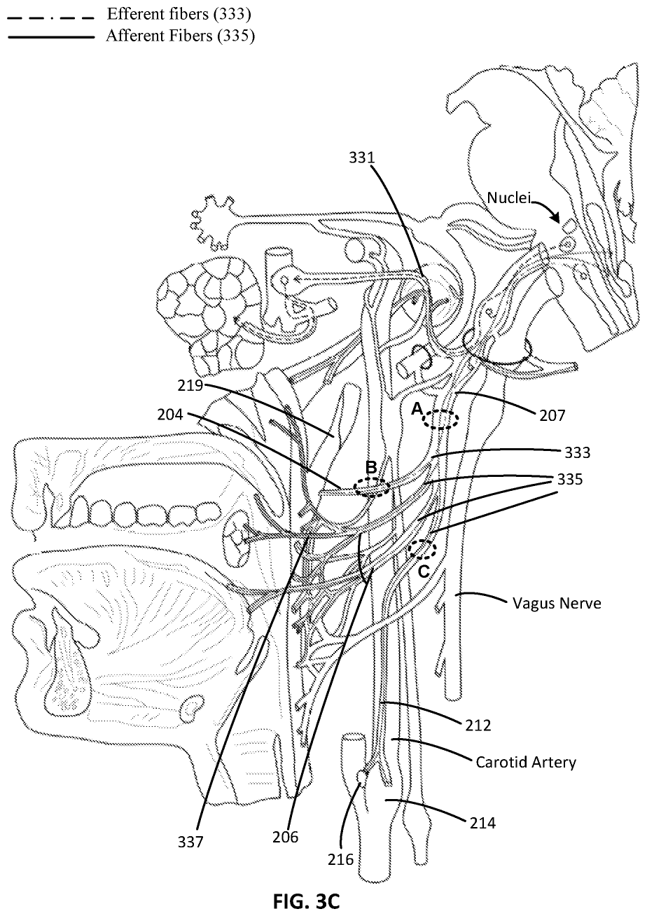

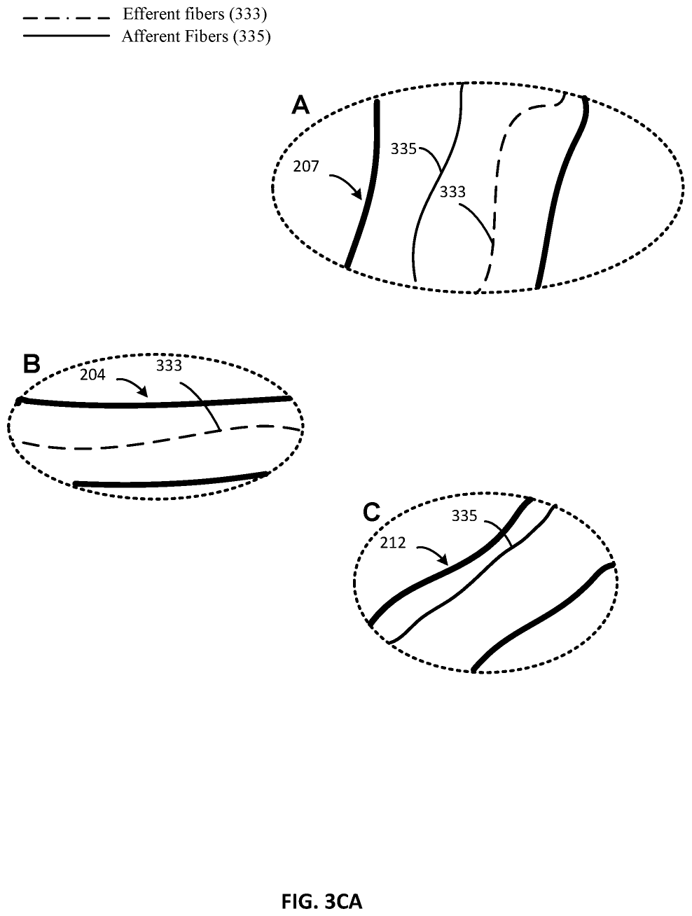

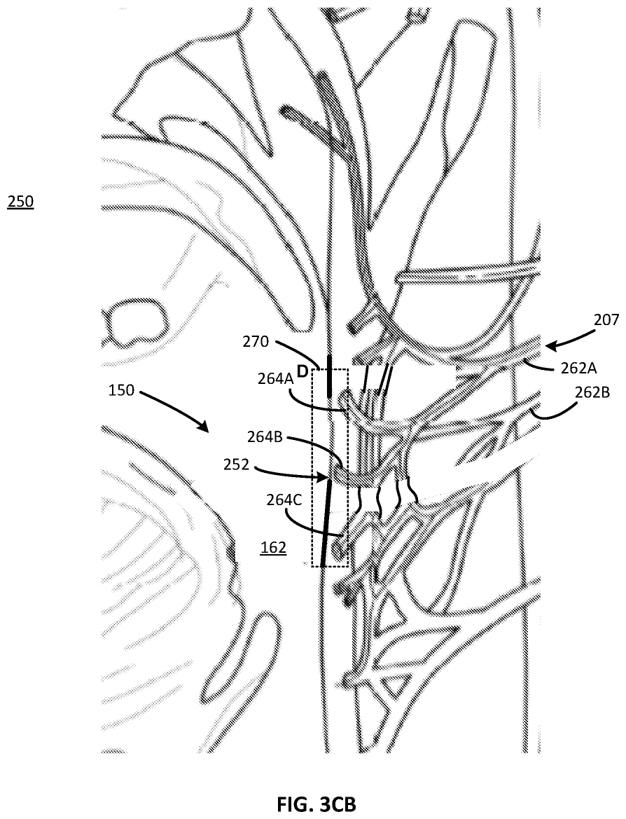

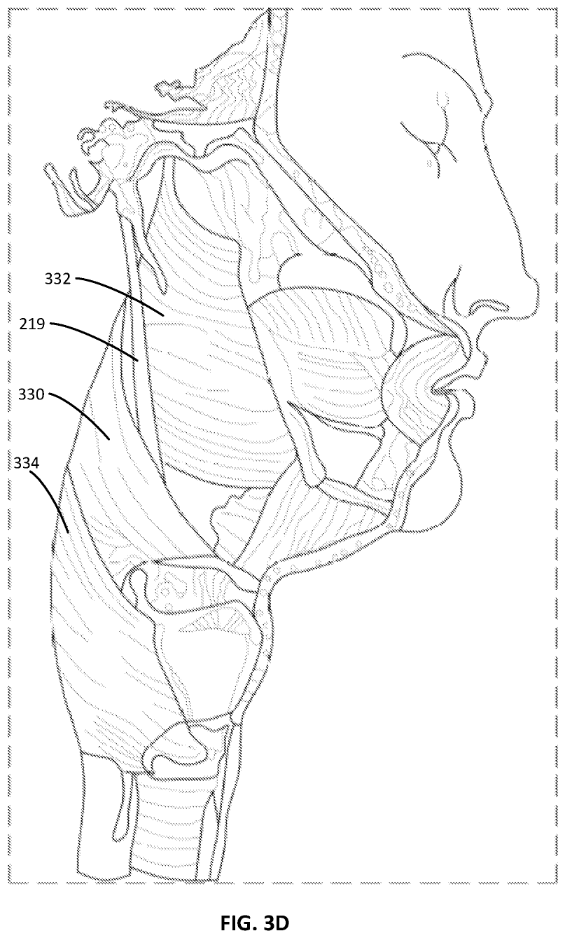

As further described herein, the upper airway includes and/or refers to air-conducting passages of the respiratory system that extend to the larynx from the openings of the noise and from the lips through the mouth, as shown generally in at least FIG. 2A and FIGS. 3A-3B. FIG. 2A depicts at least an oropharynx portion 162 of the upper airway with other aspects of the upper airway further described later.

Accordingly, in order to provide a richer context for the discussion of FIG. 2A, at least some of the nerves and/or other structures supporting, guiding, directing functions of the upper airway (and particularly at least the oropharynx portion) will be described with reference to at least FIGS. 3A-3D.

With this in mind, the glossopharyngeal nerve, sometimes referred to as the “ninth cranial nerve (CN IX)”, is a cranial nerve that connects to the brainstem from the sides of the upper medulla, anterior to the vagus nerve, and connects to various organs, muscles, and other structures in the mouth and throat, such as the carotid sinus and carotid sinus body, the middle ear, the parotid gland, the tongue, the tonsils, and the stylopharyngeus muscle, among other structures, as further illustrated by FIGS. 3A-3D. For example, and referring to FIG. 3C from the brainstem, the glossopharyngeal nerve 207 travels inferiorly down the neck near and/or alongside the jugular vein, goes behind or posterior to the styloid process before extending anterior and touching (including innervating) the stylopharyngeus muscle 219 and then extending under the hypoglossal nerve. As further described herein, the glossopharyngeal nerve 207 includes both afferent fibers 335 and efferent fibers 333, and may be referred as “a mixed nerve”. For example, the glossopharyngeal nerve 207 carries afferent sensory information to provide sensory functions via afferent fibers 335 and carries efferent motor information to provide motor functions via efferent fibers 333. The sensory functions include providing sensation, such as detecting taste, touch, and temperature, and which may cause motor functions. The motor function include enabling muscle movement, which may be responsive to sensory information.

Example sensory functions of the glossopharyngeal nerve 207 include receiving sensory information from the tonsils, the pharynx, the middle ear, the tongue, carotid sinus body 216, carotid sinus 214, including but not limited to information such as taste, sound, carbon dioxide (CO₂) levels, and oxygen levels. In some examples, the sensory information received by the afferent fibers 335 of the glossopharyngeal nerve 207 may be processed by the brain and cause motor functions, such as various reflex activity. Example reflex activity includes opening of the upper airway, gagging, coughing, and swallowing, among other reflex activity.

As a specific sensory function, a branch of the glossopharyngeal nerve 207 (e.g., branch 212) may couple to a carotid sinus body 216 and carotid sinus 214 of the carotid artery. The carotid sinus body 216 of the patient may act as a CO₂ sensor and may sense CO₂ levels in the blood (e.g., partial CO₂ measurements). In response the CO₂ levels being above a threshold, indicating insufficient oxygen is being received by the patient, the carotid sinus body 216 activates a sensory pathway associated with upper airway patency and which causes reflex activity comprising at least reflex opening of the upper airway, which is sometimes herein referred to as “reflex opening activity”. The reflex opening may cause increased patency of at least the oropharynx portion 162, among increased patency of at least some other portions of the upper airway. In some examples and/or in addition, the sensor function may include stretching of the posterior pharyngeal wall of the upper airway such that upper airway obstruction can be detected.

The sensory pathway may include the glossopharyngeal nerve 207. For example, at least one afferent fiber 335 of the glossopharyngeal nerve 207 extends to and through the branch 212 of the glossopharyngeal nerve 207 coupled to the carotid sinus body 216 (sometimes referred to as the “carotid sinus branch” or the “Hering's nerve or branch”) and carries the sensory information indicative of CO₂ levels from the chemoreceptor of or associated with the carotid sinus body 216 to the brain. The brain responds to the sensory information received by sending signals to other motor fibers and/or nerves to cause control of CO₂ levels in the blood. More particularly, in response to sensed CO₂ levels, the brain activates at least the hypoglossal nerve, innervating at least protrusion muscles of a genioglossus muscle and to cause protrusion of the tongue of the patient, thereby maintaining or increasing upper airway patency such as in at least the oropharynx portion of the upper airway.

Other sensory pathways involving the glossopharyngeal nerve 207 exist. For example, at least one afferent fiber 335 of the glossopharyngeal nerve 207 may extend from mechanoreceptors of or associated with the posterior pharyngeal wall to the brain, and carry sensory information indicative of stretching (e.g., increase/decrease) at posterior pharyngeal wall of upper airway. The brain responds to the sensory information received from the mechanoreceptors by sending signals to other motor fibers and/or nerves to cause activation of at least one upper airway patency-related muscle, such as activating an array of upper airway patency-related muscles.

In some examples, the carotid sinus 214, which is a bundle of nerve endings near the carotid sinus body 216, includes pharyngeal baroreceptors (e.g., mechanoreceptors). The pharyngeal baroreceptors are stretch receptors that are sensitive to or sense pressure changes in arterial blood pressure. In some examples, at least one afferent fiber 335 of the glossopharyngeal nerve 207 extends to and through the branch 212 of the glossopharyngeal nerve 207 coupled to the carotid sinus 214 and carries the sensory information indicative of arterial blood pressure changes from the pharyngeal baroreceptors of or associated with the carotid sinus 214 to the brain. More particularly, in response to sensed arterial blood pressure changes, without being strictly bound by theory and/or a particular mechanism of action, it is believed the brain activates at least some upper airway patency-related related muscles which cause reflex opening of the airway and cause increases or decreases in blood pressure. For example, in response to the sensory information from the pharyngeal baroreceptors indicating blood pressure changes, the brain may activate the pharyngeal constrictor muscles and/or the stylopharngeus muscles. The activation of the pharyngeal constrictor muscles may cause the pharyngeal constrictor muscles to stiffen and to maintain the upper airway open, which may be independent of other muscle activation. The stylopharngeus muscles may assist with other muscles in opening and/or maintaining upper airway opening.

Example motor functions of the glossopharyngeal nerve 207 include providing innervation to the stylopharyngeus muscle 219 and providing innervation (at least in part) to the pharyngeal plexus 337, including pharyngeal constrictor muscles. For example, contraction of the stylopharyngeus muscle 219 may increase opening of the upper airway (e.g., cause movement of the pharyngeal wall). As another example, contracting the at least one pharyngeal constrictor muscle may stiffen the upper airway (e.g., increases pharyngeal muscle tone) to reduce collapsibility of the upper airway.

Referring back to FIG. 1, in some examples and at 12, the selective stimulation of the at least one fiber of the glossopharyngeal nerve may include stimulating the at least one fiber of the glossopharyngeal nerve, while not stimulating, or stimulating below a threshold level, other fibers of the glossopharyngeal nerve. The selective stimulation of the glossopharyngeal nerve may be used to selectively stimulate specific muscle and/or to activate or prime a sensory pathway associated with a reflex opening activity, without causing reflex activity associated with coughing and/or closing of the trachea. In some examples, the selective stimulation may include selective stimulation of specific afferent fiber(s) and/or efferent fiber(s), while not stimulating other afferent and/or efferent fibers, such as via steering (e.g., current steering). Steering may include developing and/or causing a particular shaped stimulation field, via the at one stimulation element, to encompass the fiber(s) of interest, while avoiding others. In some examples, the selective stimulation may be provided by selecting a target location along a length of the glossopharyngeal nerve (for applying the stimulation), such as more distal locations at which the respective afferent and/or efferent nerve fibers may already be isolated or mostly isolated from each other within different/distinct branches of the glossopharyngeal nerve. In some examples, the target location of the glossopharyngeal nerve may be more proximally located and/or the target nerve fibers/bundles (at the target location) may be selectively stimulated via activating a particular combination of electrodes of an array of stimulation electrodes arranged within/along a cuff electrode, axial electrode array, etc. In some examples, the selective stimulation may include selecting and/or adjusting the timing, strength, shape of the stimulation to stimulate the muscle and/or activate or prime a sensory pathway without causing reflex activity associated with coughing and/or closing of the trachea.

In some examples, the selective stimulation may be adjusted non-invasively by a clinician through programming electrode configurations and electrical thresholds. In some examples, the selective stimulation may include auto-titration.

While a distal location of glossopharyngeal nerve may be used to provide the selective stimulation, in some examples, a proximal location of the glossopharyngeal nerve may be used to provide selective stimulation due to greater ease of accessing the proximal location and/or implanting at least a portion of a stimulation element as compared to the distal location.

In some examples, stimulating the glossopharyngeal-related tissue may be used for treating SDB, such as for sleep apnea. Sleep apnea generally refers to the cessation of breathing during sleep. One type of sleep apnea, referred to as OSA, is characterized by repetitive pauses in breathing during sleep due to the obstruction and/or collapse of the upper airway, and is usually accompanied by a reduction in blood oxygenation saturation, and thus increased CO₂ levels in the blood. During sleep, the upper airway patency-related muscles may not function properly as the muscles become more relaxed, which may cause breathing obstruction as tissue closes in and blocks the upper airway. In some examples, electrical stimulation of glossopharyngeal nerve may activate or prime a sensory pathway associated with neural control of upper airway patency, which may cause a neural response leading to contraction of (or a tone response of) upper airway patency-related muscle to improve or maintain upper airway patency. In some examples, the electrical stimulation of the glossopharyngeal-related tissue may active other motor functions and/or direct activate the stylopharyngeus muscle as further described herein.

As used herein, upper airway patency-related muscles include and/or refer to muscles associated with respiration, and which may be involved with upper airway patency. In some examples, the upper airway patency includes patency of at least the oropharynx portion of the upper airway. In some examples, upper airway patency-related muscles include the stylopharyngeus muscle and pharyngeal constrictor muscles, which are innervated by the glossopharyngeal nerve. Some example upper airway patency-related muscles include the genioglossus muscle, which is innervated by the hypoglossal nerve. Further example upper airway patency-related muscles include infrahyoid strap muscles, e.g., sternohyoid, sternothyroid, thyrohyoid, and/or omohyoid muscles, which are innervated by an infrahyoid strap muscle-innervating nerve. In some examples, the IHM-innervating nerves comprise those nerve branches which innervate (directly or indirectly) at least one of the respective infrahyoid strap muscles with such nerve branches being distinct from the ansa cervicalis nerve loop (e.g. including the superior root and inferior root) from which they extend. Examples are not so limited, and in some instances, upper airway patency-related muscles may comprise other muscles, innervated by respective nerves.

In some examples, the at least one fiber of the glossopharyngeal nerve comprises an afferent fiber. For treatment of SDB, in some examples, increased respiratory effort resulting from the difficulty in breathing through an obstructed airway is avoided or mitigated by selectively stimulating the at least one afferent fiber of the glossopharyngeal nerve to cause reflex opening of the upper airway and/or to prime a sensory pathway associated with reflex opening activity. Priming a sensory pathway may include and/or refer to stimulating an afferent fiber such that a sensitivity of the afferent fiber to sensory information is increased. In some examples, priming a sensory pathway may be referred to as gating the reflex opening activity or response, such as gating the airway response to partial pressure of CO₂. For example, in response to the stimulation, the afferent fiber is more sensitive to the sensory information indicative of CO₂ levels in the blood and/or identifies the received sensory information with an increased value. As described above, the sensory pathway associated with reflex opening activity may be associated with sensed CO₂ levels in the blood, and may cause or otherwise be associated with activation of at least one upper airway patency-related nerve (e.g., hypoglossal nerve) to cause at least one upper airway patency-related muscle to contract. In one non-limiting example, activation of the hypoglossal nerve will cause contraction of the genioglossus muscle, which results in protrusion of the tongue of the patient, thereby increasing upper airway patency.

However, one aspect of implementing therapy via stimulating the sensor pathway(s) to invoke a reflex opening activity is that not just one upper airway patency-related nerve becomes activated/contracts, but the entire array (or substantially the entire array) of such upper airway patency-related muscles become activated/contract which may otherwise be impractical because of the number and types of locations at which a stimulation element would have to be implanted for each respective upper airway patency-related muscle responding as part of the reflex opening, because of the coordination of stimulating many different nerves/muscles, because of the power requirements of doing so, and other difficult challenges if one were to attempt stimulating the entire array of upper airway patency-related nerves and/or muscles. Instead, by stimulating a single nerve (or portion thereof), namely the glossopharyngeal nerve, via its sensory pathway, the example therapy may invoke a comprehensive response of most or all of the upper airway patency-related muscles as part of the reflex opening activity. This example arrangement stands in contrast to merely stimulating one or two such upper airway patency-related nerves/muscles resulting in isolated muscles responses, or instead of an impractical attempt to separately stimulate all the upper airway patency-related nerves (and/or associated muscles) that otherwise occurs naturally via a reflex opening activity.

Moreover, for some patients who receive a therapy in which just one upper airway patency-related nerve (or at most two different nerves) may be stimulated, there may be uncertainty about whether the attempted therapy will be successful in view of the uncertainty about the effectiveness of the particular nerve selection (or particular branch selection), of the type of stimulation element, of the selected stimulation energy settings, of patient criteria, and/or of other factors.

In accordance with at least some or most of these uncertainties may be avoided or mitigated via examples of the present disclosure in which stimulation of a sensory nerve/pathway of the glossopharyngeal nerve is performed to invoke a reflex opening activity of the upper airway to increase or maintain its patency. Accordingly, by increasing the sensitivity to CO₂ blood levels, the example devices, apparatus, and/or methods may increase the likelihood of reflex opening of the upper airway

In some examples, the at least one fiber comprises an efferent fiber. In some examples and for treatment of SDB, increased respiratory effort resulting from the difficulty in breathing through an obstructed upper airway is avoided or mitigated by selectively stimulating the at least one efferent fiber of the glossopharyngeal nerve to hold the airway open during at least a portion of the respiratory cycle, such as a portion of the inspiratory phase of breathing. In some examples, the stimulation may be timed relative to the inspiratory phase of breathing. The selective stimulation may cause contraction (e.g., full contraction or tone response) of the stylopharyngeus muscle and/or at least one pharyngeal constrictor muscle to stiffen the upper airway and to reduce collapsibility of the upper airway.

In some examples, the at least one afferent fiber and at least one efferent fiber are stimulated at the same time and/or different times to activate sensory function(s) and/or motor function(s) of the glossopharyngeal nerve. For example, efferent fibers and at least one (e.g., a portion) of the afferent fibers of the glossopharyngeal nerve may be stimulated, while not stimulating other afferent fibers. In some such examples, the stimulation of the at least one efferent fiber may be timed with respiration information and the at least one afferent fiber may be stimulated independent of the respiration information using a stimulation energy level below a threshold (e.g., low tonic as further described herein). In some examples, the efferent fiber(s) may be stimulated using a stimulation energy level above a threshold (e.g., a suprathreshold).

In any of the above-described examples, the selective stimulation of at least one afferent fiber and/or at least one efferent fiber may further comprise preventing or mitigating the reflex activity of coughing and/or closing of the trachea. Both reflex activity of coughing and/or closing of the trachea may be activated by sensory information carried by particular afferent fibers of the pharyngeal, tonsillar, and/or lingual branches of the glossopharyngeal nerve. To avoid causing reflex activity of coughing and/or closing of the trachea, the stimulation may be selectively applied to respective afferent fiber(s) and/or efferent fiber(s) (of the glossopharyngeal nerve) which are associated with reflex opening activity of upper airway patency and/or to activate upper airway patency-related muscles. The respective fiber(s) may be selectively stimulated while not stimulating, or by applying stimulation below a threshold level (e.g., tonic stimulation is below a suprathreshold at which contraction occurs) to cause priming or activation of the sensory pathway, other fibers of the glossopharyngeal nerve. The other fibers may include the particular afferent fiber(s) associated with sensory pathways of reflex activities of coughing and/or closing of the trachea. By providing the selective stimulation, upper airway patency may be increased or maintained without causing (or with mitigating) unwanted reflex activity, associated with stimulating the glossopharyngeal nerve, such as coughing. In some such examples, the selective stimulation may be used to treat patients that are non-responsive and/or non-compliant with other types of SDB treatment. As used herein, stimulating, or activating muscle may cause (or include) contraction of the muscle.

In some examples, the selective stimulation, which may be referred to as controlled stimulation, may include or additionally include controlling when (e.g., timing or in response to SBD-related parameters) the stimulation is applied, stimulation levels, number of stimulations, and/or pace, among other variables. For example, and in accordance with various examples of the method 10, the stimulation may be applied in response to at least one SDB event, as a plurality of stimulations at a duty cycle, and/or as an electrical stimulation applied during a treatment period which is below a threshold level (e.g., steady stimulation during or over the treatment period). In some examples, once the treatment period begins, the stimulation may be applied throughout the treatment period (e.g., nightly) until the treatment period is over. In such examples, the stimulation is applied regardless of the number of SDB events occurring for the patient.

As further described herein, the method 10 may comprise a number of additional steps and/or variations, such as those illustrated in connection with FIGS. 2A-10. Such example variations may include selectively stimulating different target locations of the glossopharyngeal nerve (or other glossopharyngeal-related tissue) and/or selectively stimulating based on at least one SDB-parameter, among other variations.

FIGS. 2A-2D illustrate an example upper airway of a patient and at least one stimulation element for stimulating a glossopharyngeal-related tissue.

FIG. 2A is a side view schematically illustrating an example upper airway of a patient. More specifically, FIG. 2A is a diagram 140 of a side sectional view (cross hatching omitted for illustrative clarity) of a head and neck region 142 of a patient. In particular, an upper airway portion 150 extends from the mouth 144 to a neck portion 155. The upper airway portion 150 includes a velum (soft palate) portion (or region) 160, an oropharynx portion (or region) 162, and an epiglottis-larynx portion (or region) 164. The velum (soft palate) portion 160 includes an area extending below sinus 161, and including the soft palate 146 approximately to the point at which tip 148 of the soft palate 146 meets a portion of tongue 147 at the back of the mouth 144. The oropharynx portion 162 extends approximately from the tip of the soft palate 146 along the base 152 of the tongue 147 until reaching approximately the tip region of the epiglottis 154. The epiglottis-larynx portion 164 extends approximately from the tip of the epiglottis 154 downwardly to a point above the esophagus 157.

FIG. 2B illustrates an example deployment of a stimulation element 201. More specifically, FIG. 2B is diagram including a front view schematically representing deployment 200 of an example stimulation element 201 in or near to a head region 203 and/or neck region 205 of the patient. The stimulation element 201 may include a stimulation electrode arrangement 211 including an electrode or an array of electrodes, among other elements which may apply electrical stimulation to tissue. As shown in FIG. 2B, the stimulation electrode arrangement 211 may be deployed at or near the glossopharyngeal nerve 207. In some examples, the stimulation electrode arrangement 211 may be implanted within or near the head region 203 and coupled to or otherwise at or near the glossopharyngeal nerve 207. While the stimulation electrode arrangement 211 is shown as being chronically implanted posterior to a face portion of the head region 203, examples may include the stimulation electrode arrangement 211 being deployed more inferiorly than illustrated by FIG. 2B, such as in the neck region 205 of the patient. In some examples, the stimulation element 201 may be external to the patient's body, but at or near the glossopharyngeal nerve 207.

In some such examples, the stimulation element 201 and/or the stimulation electrode arrangement 211 may form part of a wearable device or patch which may be placed at or near the glossopharyngeal nerve 207. In some examples, the stimulation element 201 and/or the stimulation electrode arrangement 211 may comprise a chronically implantable element such as (but not limited to) a cuff electrode to at least partially enclose at least a portion of the glossopharyngeal nerve 207 (or other glossopharyngeal-related tissue) at a target location or such as an axial electrode array or other electrode carrier configurations. In some examples, the stimulation electrode arrangement 211 may be anchored to non-nerve tissue at or near the at least one fiber of the glossopharyngeal nerve 207. In some examples, other glossopharyngeal-related tissue may be stimulated.

In some examples, the stimulation element 201 forms part of a medical device and further includes a pulse generator (PG) 210. In some examples, the PG 210 may be an implantable pulse generator (IPG). In some examples, the PG 210 is external to the patient's body, as shown by FIG. 2B. The PG 210 may supply electrical signals to the stimulation electrode of the stimulation electrode arrangement 211, which cause the stimulation electrode arrangement 211 to apply electrical stimulation to at least one fiber of the glossopharyngeal nerve 207, such as performing the method 10 of FIG. 1.

FIG. 2C is a block diagram schematically representing an example device which may be used to implement the method 10 of FIG. 1 and/or stimulate glossopharyngeal-related tissue. Various aspects of stimulation locations, accessing the stimulation locations, control of the stimulation, and glossopharyngeal-related tissue 213 are further described in associated with at least FIGS. 2D-15. Among these examples, at least stimulation portion 1600 in FIG. 10 provides a general framework for various examples and types of stimulation, as further described later, relative to which the examples of FIG. 2C may be further appreciated.

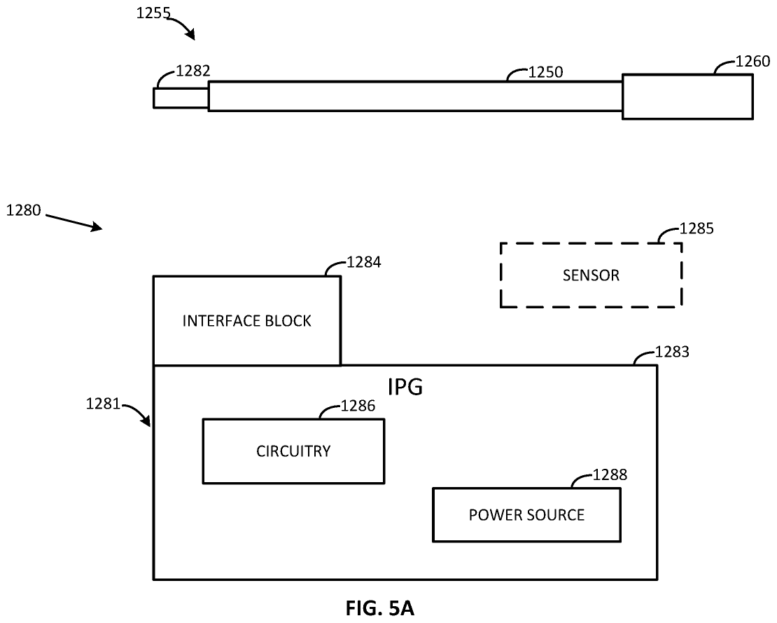

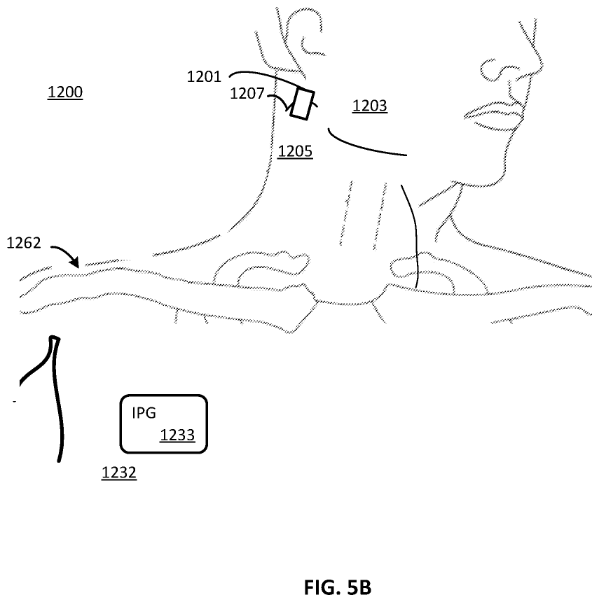

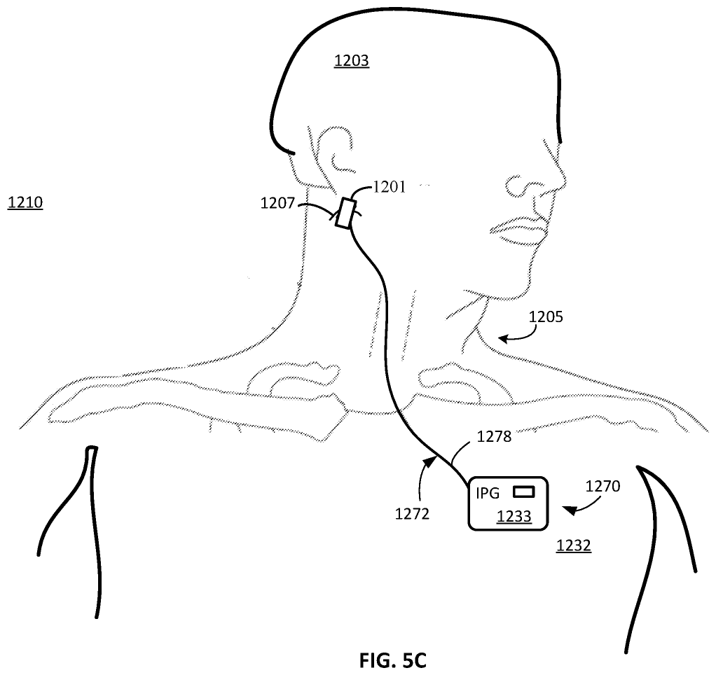

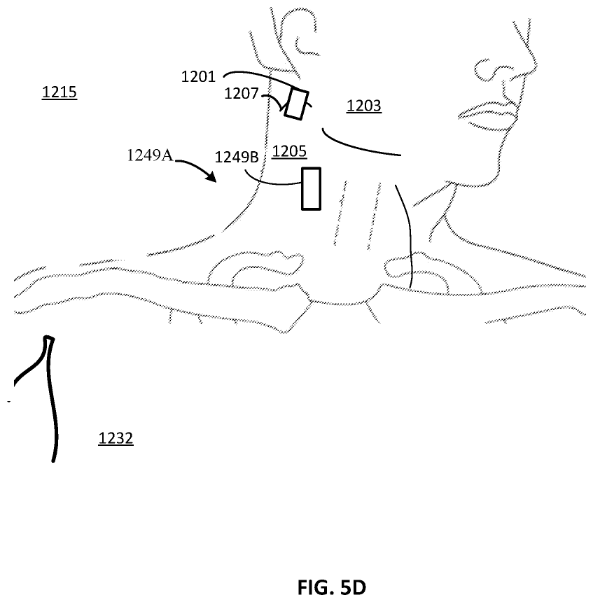

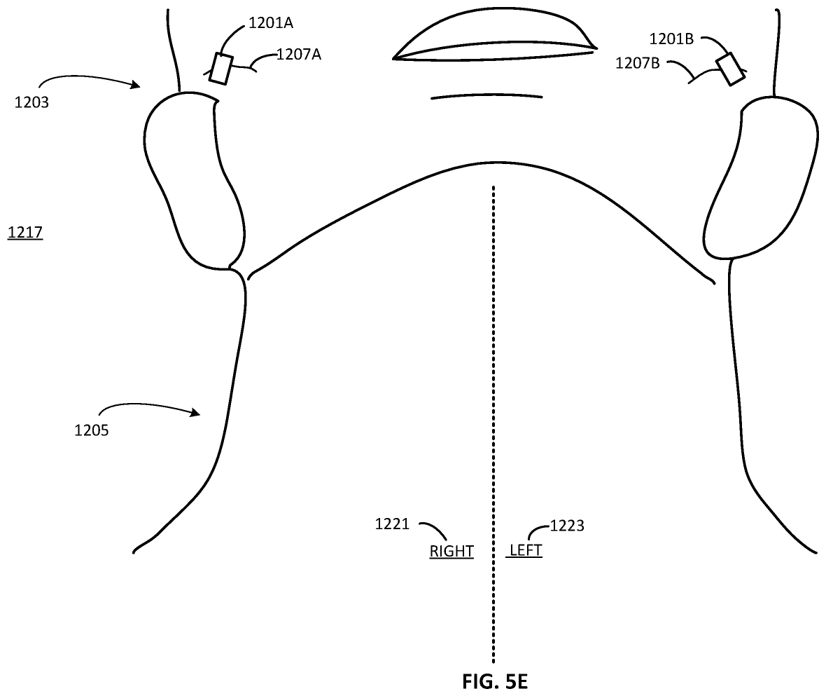

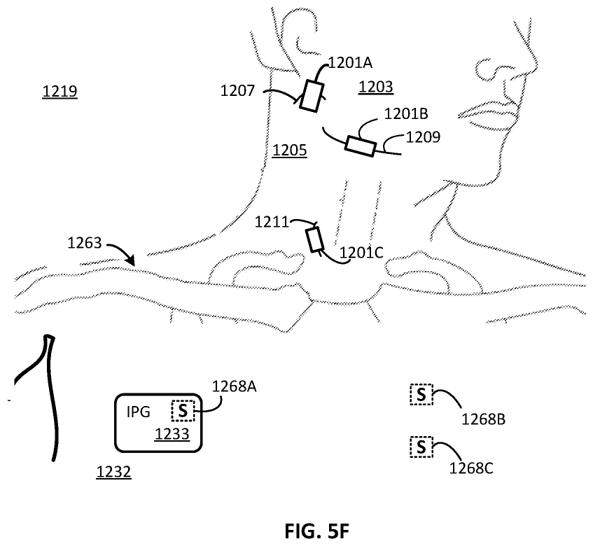

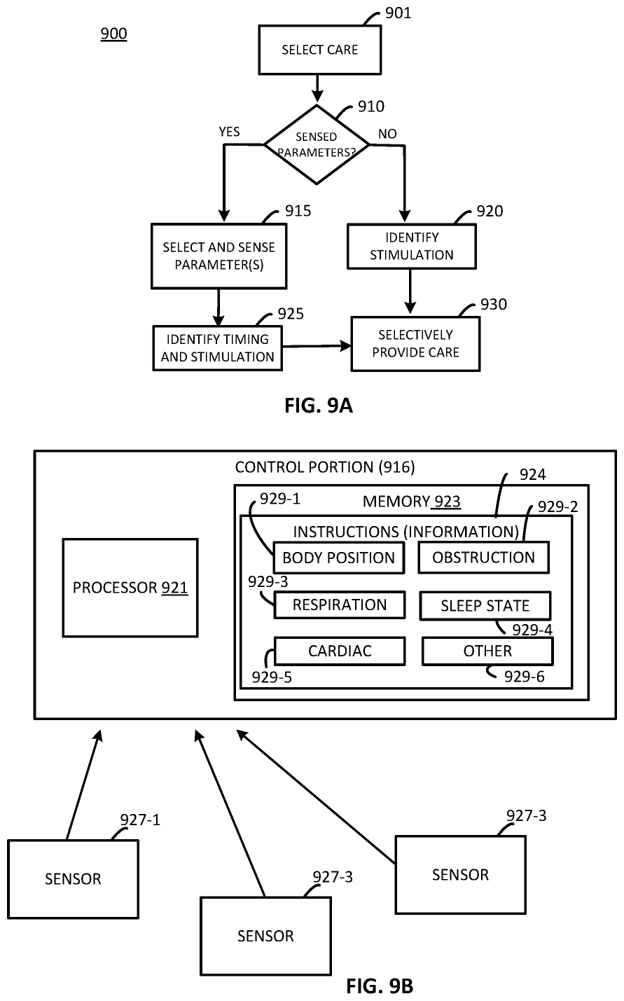

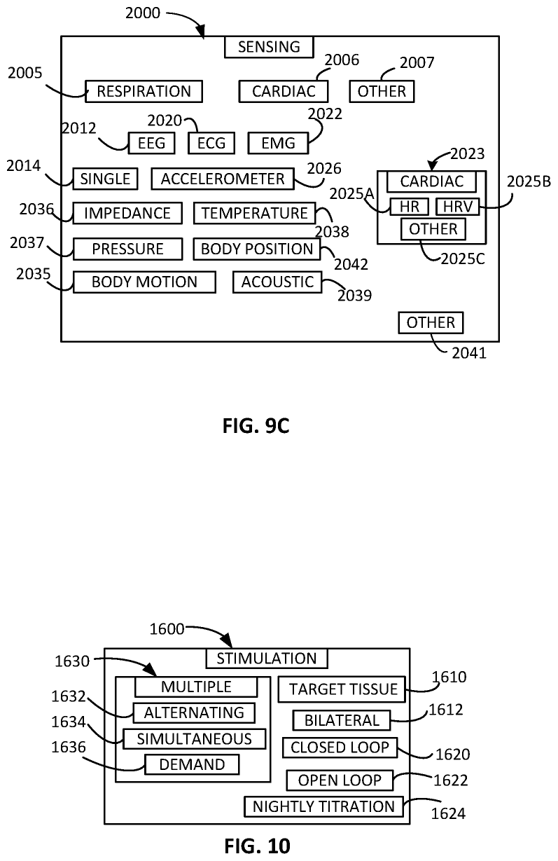

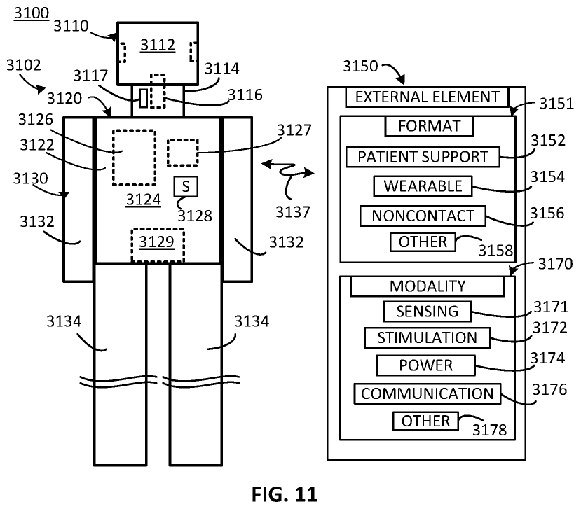

As shown in FIG. 2C, in some examples, a device 215 may comprise a stimulation element 201. In some examples, the stimulation element 201 may include a stimulation electrode arrangement, such as at least one stimulation electrode. In some examples, the stimulation element 201 may further include a lead that supports at least one stimulation electrode (e.g., of a stimulation electrode arrangement) of the stimulation element and include a stimulation support portion (e.g., 133 in FIG. 2D). As described in association with at least FIG. 2D, among other example implementations, one example implementation of a stimulation support portion may comprise stimulation (or control) circuitry, which may be embodied as a pulse generator (PG) or implantable pulse generator (IPG). Further example implementations of a stimulation support portion may comprise a sensing element to perform sensing and/or to receive sensed data from sensors external to the stimulation element (e.g., including being external to the stimulation support portion), with such sensors being implantable and/or external to the body. In some examples, the sensor(s) may comprise at least some of substantially the same features as described throughout FIGS. 5A-15, with particular reference to sensor portion 2000 of FIG. 9C and/or external element 3150 in FIG. 11.

With further reference to FIG. 2C, in some examples, the stimulation element 201 may form part of a catheter or lead which is placed within the body. Various types of leads may be used, including but not limited to, a spiral-type lead, a basket or lasso type lead, and a lead with tined tips, among others. In general terms, in some examples, the stimulation element 201 (or at least a portion thereof) is located at a position adjacent to glossopharyngeal-related tissue 213 such as (but not limited to) the glossopharyngeal nerve such that stimulation applied via the stimulation element 201 is delivered to the glossopharyngeal nerve. Via this example arrangement, the stimulation element 201 becomes positioned into stimulating relation to the target glossopharyngeal-related tissue 213. In some examples, “stimulating relation” may include and/or refer to a stimulation element 201 (e.g., at least one electrode) being in a position, orientation, and/or distance such that the applied stimulation signal provides at least some capture of a nerve (e.g., at least tone response of muscle, and, in some instances, supra-threshold or full muscle contraction) and/or of a muscle. In some instances, the stimulation may be tonic stimulation, as further described herein.

In some examples, stimulation element 201 may comprise at least one stimulation electrode(s) which may take a wide variety of forms, and may be incorporated within a wide variety of different types of stimulation electrode arrangements, at least some of which are described in association with at least FIGS. 5A-5F. In some examples, the stimulation element 201 includes a pair of electrodes or a plurality of pairs of electrodes. In some examples, the stimulation element 201 includes a plurality of ring electrodes. In other examples, the stimulation element 201 includes a planar electrode or a plurality of planar electrodes. In some examples, the stimulation applied may be bipolar or monopolar.

In some examples, the electrode(s) of the stimulation element 201 used for applying stimulation also may be used for sensing, but not necessarily for simultaneous stimulation and sensing. However, in some examples, the electrode(s) of the stimulation element 201 are used solely for applying stimulation while some electrode(s) may be used solely for sensing.

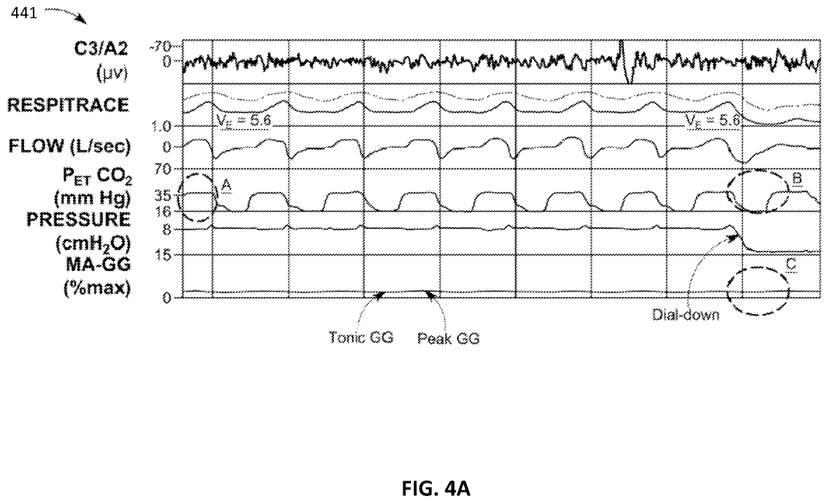

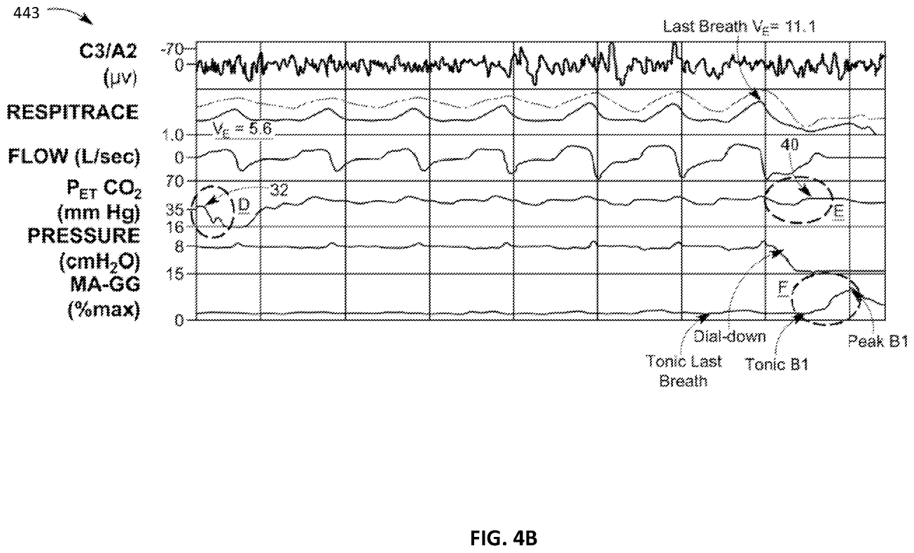

In some examples, the device 215 may be implanted within the patient's body. For example, the stimulation element 201, or at least a portion thereof, may be inserted within the patient's body and maneuvered to the target location for applying stimulation to the glossopharyngeal-related tissue 213, as further described in connection with at least FIGS. 3A-4B. In some examples and as noted above, the stimulation element 201 of the device 215 may further include a lead that supports the at least one stimulation electrode.

In some examples, as noted above, the stimulation element 201 may further include a stimulation support portion (e.g., at least 133 in FIG. 2D) which may be embodied as a pulse generator, such as illustrated in connection with at least FIGS. 5A-5F. In some such examples, the entire pulse generator (and/or other power, control, and/or communication elements) may be implantable while in some examples, some portions of the pulse generator (and/or other power, control, and/or communication elements) may be external to the patient as further described in association with at least FIG. 11. In some examples, the IPG or a non-implanted pulse generator may be separate from the stimulation electrode arrangement. In some examples, the pulse generator may be located within the head-and-neck region or the pectoral region of the patient. In some examples, the IPG may be chronically implanted in at least one of the torso region, the neck region, or the cranial region. The torso region may include the sternum, pectoral region, or other areas. The neck region may include the neck and other areas, such as a transitional area of the neck (e.g., between the neck and torso, and/or between the neck and cranial region) including the clavicle, manubrium (e.g., at top of sternum), and mandible. In some examples, the cranial region may include the skull, such as behind the ear of the patient, among other locations. In some examples, components may be implanted in the cranial region or in the head region, which may be referred to as a “head-and-neck region” for ease of reference.

In some examples, the stimulation element 201 may include a stimulation support portion, such as further described herein in connection with at least FIG. 2D. As further described herein, in some examples the stimulation support portion may be implemented as a pulse generator, such as an IPG.

As shown in FIG. 2D, in some examples, the stimulation support portion 133 may comprise stimulation function circuitry 134A, a power element 134B, a sensing element 134C, a control element 134D, a communication element 134E (e.g., at least a receiver), and/or other element 134F.

In some examples, the stimulation function circuitry 134A may comprise passive stimulation circuitry, e.g., circuitry which does not generate a stimulation signal but which may receive a stimulation signal generated elsewhere (e.g., external of the patient or from an implanted device) and which is then communicated (e.g., via lead) to the electrodes of the stimulation electrode arrangement for stimulating the glossopharyngeal-related tissue 213 and/or other upper airway patency-related tissue.

With further reference to the particular example illustrated in FIG. 2D, in some examples, the stimulation function circuitry 134A comprises active stimulation circuitry, e.g., components sufficient to generate a stimulation signal within the stimulation support portion 133 for transmission (e.g., via a lead or other means) to the electrodes of the stimulation electrode arrangement of the stimulation element (e.g., 201 of FIGS. 2B-2C). In some such examples, the stimulation support portion 133 may sometimes comprise and/or be referred to as a pulse generator (PG). Moreover, in some such examples, given the stimulation support portion 133 being sized and shaped for implantation in the head-and-neck region, the stimulation support portion 133 may sometimes be referred to as a microstimulator.

Whether referred to as a microstimulator or not, in these examples the housing 135 of the stimulation support portion 133 may sealingly contain (e.g., encapsulate) the stimulation function circuitry 134A, along with other elements such a power element 134B, communication element 134E, and/or control element 134D, among other potential components (e.g., sensing 134C, etc.).

In some examples, the stimulation support portion 133 of the stimulation element 201 may comprise a power element 134B. The power element 134B may be non-rechargeable, in some examples. However, the power element 134B may be re-chargeable in some examples such that the power element 134B receives power from a power source external of the stimulation support portion 133, with the power source being implantable in some examples or being external of the patient in some examples. For instance, the power element 134B may receive power via a wired connection (e.g., in some examples in which the power source is implantable) or via wireless communication, in which the power source may be implantable or external to the patient. In some examples in which the power source may be external to the patient, the power source may comprise at least some of substantially the same features and attributes as external power portion 3174 in FIG. 11, as further described below.

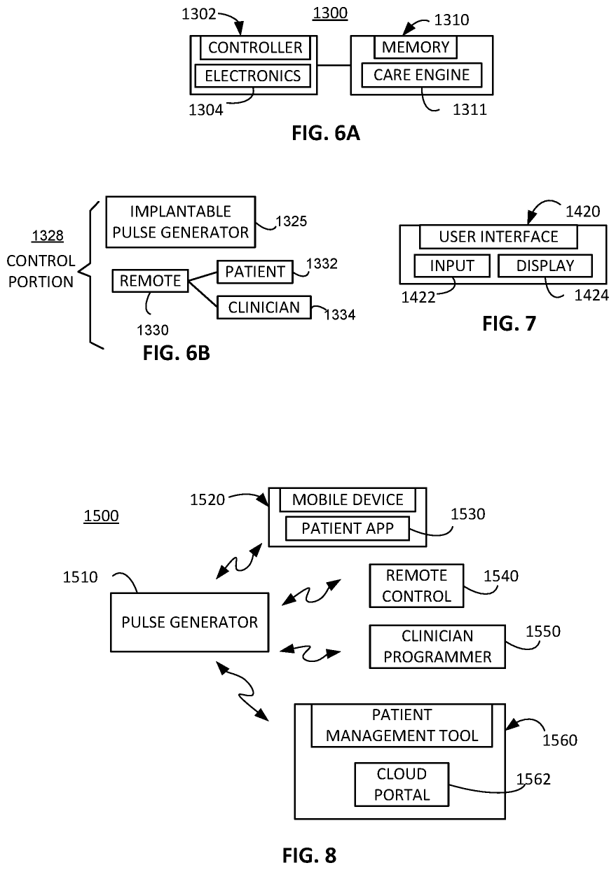

In some examples, the stimulation support portion 133 comprises a control element 134D which provides on-board control of at least some of the functions of the stimulation element 201 (including stimulation electrode arrangement 211, stimulation support portion 133, and/or other components of the stimulation element 201). In some examples, the control element 134D may comprise the entire control portion for the stimulation element 201. In some examples, the control element 134D may form part of a larger control portion in which the control element 134D may receive at least some control signals from components of the control portion external to the stimulation support portion 133. In some such examples, these components of the control portion which are external to the stimulation support portion 133 also may be external to the patient. For example, the control element 134D of stimulation support portion 133 may comprise at least a partial implementation of, and/or communicate with, a control portion 1300 or 1328 of FIGS. 6A-6B, a control portion 1916 of FIG. 9B, stimulation portion 1600 of FIG. 10, and/or control portion 2500 of FIG. 14. As such, consistent with the later described control portion 1300 of FIG. 6A, the control element 134D in FIG. 2D also may comprise a memory to store stimulation therapy information (e.g., therapy settings, usage, outcomes, etc.), control information, sensed information (per sensing element 1034C), etc.

In some examples, the sensing element 134C of stimulation support portion 133 may store data sensed by an on-board sensor of the stimulation element 201 and/or sensed via sensor external to the stimulation element 201 (e.g., external to stimulation support portion 133, stimulation electrode arrangement) with such sensor (external to the stimulation element 201) being implantable or external to patient. In some examples, an on-board sensor may comprise an accelerometer (e.g., tri-axis), gyroscope, etc. In some examples, such on-board sensor may comprise an electrode exposed on surface of housing, which in combination with other electrodes may be used to sense impedance and/or other biosignals. With these brief examples in mind, it will be understood that in some examples the sensing element 134C may comprise, and/or receive sensed information from, at least some of substantially the same sensing elements, functions, etc. as later described in association with at least FIG. 6A (e.g., control portion 1300, care engine 1311), FIG. 9B (e.g., control portion 916), FIG. 9C (e.g., sensing portion 2000), FIG. 10 (e.g., stimulation portion 1600), and/or FIG. 11 (e.g., external element 3150).

In some examples, the stimulation support portion 133 of the stimulation element 201 may comprise a communication element (e.g., coil, antenna, and any related circuitry) to transmit and/or receive the control information, therapy data, sensed data, and the like. In addition to, or instead of these examples, the communication element may be configured to facilitate receive power from a power source(s) external to the stimulation support portion 133, whether via wired connection or wirelessly. In some examples, the communication element 134E may be implemented via various forms of radiofrequency communication and/or other forms of wireless communication, such as (but not limited to) magnetic induction telemetry, Bluetooth (BT), Bluetooth Low Energy (BLE), near infrared (NIF), near-field protocols, Wi-Fi, Ultra-Wideband (UWB), ultrasound, and/or other short range or long range wireless communication protocols suitable for use in communicating between implanted components within the body and/or communicating between implanted components and external components in a medical device environment.

It will be understood that in some examples of the present disclosure, a lead may be omitted and at least some of the operative components of the stimulation support portion 133 may be incorporated into and/or with the stimulation electrode arrangement, such as illustrated by (but not limited to) the example stimulation electrode arrangement 1201 of FIG. 5D. In some such examples, the stimulation electrode arrangement may sometimes comprise, or be referred to as, a leadless stimulation electrode arrangement or a leadless stimulation element 201. In some of these examples, the functions and/or components of the stimulation support portion 133 which are incorporated into the stimulation electrode arrangement may comprise passive stimulation circuitry (which may be embodied as a part of the communication element 134E) to receive a stimulation signal generated elsewhere and conduct this stimulation signal to the electrodes of the stimulation electrode arrangement.

Referring back to FIG. 2C, in some examples, portions of the stimulation element 201 may comprise anchoring elements which act to maintain at least the stimulation electrode arrangement in a selected location to maintain at least some electrodes of the stimulation element 201 in stimulating relation to glossopharyngeal-related tissue 213 or other tissue. Accordingly, in some such examples, the housing of stimulation support portion (133 of FIG. 2D) may comprise an array of anchor elements, which may comprise tines, barbs, and/or other tissue-engaging structures to hinder or prevent movement of the housing relative to target tissue in which the stimulation support portion is present. In some examples, with or without such tines, barbs, etc., a shape of the housing of the stimulation support portion may act as, and/or form part of, an anchoring arrangement.

FIGS. 3A-3D illustrate example target locations for stimulation of the glossopharyngeal-related tissue and surrounding anatomy, such as for implementing the method 10 of FIG. 1. In various examples, at least a portion of the stimulation element 201 of FIG. 2B-2D may be deployed at any of the example target locations A, B, C illustrated by FIG. 3A and/or used to implement the method 10 of FIG. 1. Furthermore, the variations described in connection with FIGS. 3A-3D may be implemented as part of the method 10 of FIG. 1.

FIG. 3A illustrates example target locations for the at least one stimulation element or portions thereof, and with respect to the upper airway anatomy as described previously in connection FIG. 2A. FIG. 3B illustrates various additional anatomy associated with the glossopharyngeal nerve 207. As shown by FIG. 3A, the example glossopharyngeal nerve 207 extends from the brainstem (the nuclei of FIG. 3B) to various structures via different branches 204, 206, 212 of the glossopharyngeal nerve 207. The branches 204, 206, 212 illustrated include the stylopharyngeus nerve branch 204, the pharyngeal, tonsillar, and lingual branches 206, and the carotid sinus branch 212.

As previously described, the glossopharyngeal nerve 207 includes both afferent fibers and efferent fibers. The afferent fibers originate at the pharynx, middle ear, posterior of the tongue 147 (including taste buds), and the carotid sinus body 216 and carotid sinus 214 of the carotid artery 218, and terminate at the medulla oblongata. The afferent fibers may travel along or otherwise be associated with the pharyngeal, tonsillar, and lingual branches 206, the carotid sinus branch 212, and the branch 331 illustrated by FIG. 3B of the glossopharyngeal nerve 207. The efferent fibers originate at the medulla oblongata and terminate at the parotid salivary gland, the glands of the posterior tongue (e.g., travel along or are otherwise associated with pharyngeal, tonsillar, and lingual branches 206), and the stylopharyngeus muscle 219 (e.g., travel along or are otherwise associated with the stylopharyngeus nerve branch 204).

FIG. 3B illustrates the various different branches 204, 206, 212, 331 of the glossopharyngeal nerve 207 and associated structures, including originating structures and terminating structures. An example afferent fiber of the carotid sinus branch 212 originates at the carotid sinus 214 and carotid sinus body 216 and terminate at the brainstem, e.g., nuclei of CN IX. An example efferent fiber of the stylopharyngeus nerve branch 204 originates at the brainstem, e.g., nuclei of CN IX, and terminate at the stylopharyngeus muscle 219.

In various examples, at least one stimulation element (e.g., stimulation element 201 of FIGS. 2B-2C) may be used to selectively stimulate at least one fiber of the glossopharyngeal nerve 207. Among other objectives, the selective stimulation may be used to prevent and/or mitigate unintentional activating of reflex activity associated with coughing and/or closing of trachea. As previously described, the at least one fiber may comprise an afferent fiber of the glossopharyngeal nerve 207, and/or an efferent fiber of the glossopharyngeal nerve 207. The selective stimulation may be used to selectively and controllable cause at least one sensory function and/or at least one motor function of the glossopharyngeal nerve 207.

Referring back to FIG. 3A, FIG. 3A illustrates different target locations A, B, C for selectively stimulating at least one fiber of the glossopharyngeal nerve 207. The different target locations A, B, C may be used to selectively activate motor functions, sensory functions, or both motor functions and sensory functions of the glossopharyngeal nerve 207.

As shown by FIG. 3A, target location A is a proximal location on a stem of the glossopharyngeal nerve 207, which may be used to selectively activate motor functions, sensory functions, or both motor functions and sensory functions of the glossopharyngeal nerve 207. Target location A includes both afferent fibers and efferent fibers of the glossopharyngeal nerve 207. To avoid or mitigate activating reflex activity of coughing and/or closing of the trachea in response to selective stimulation at target location A, example methods may specifically select afferent fibers and/or efferent fibers to be stimulated, while other afferent and/or efferent fibers are not stimulated or are stimulated below a threshold level that activates particular (unwanted) reflex activity.

In association with at least target A (and target B as well as other target locations), example motor functions of the glossopharyngeal nerve 207 include pharyngeal constrictor muscle(s) (e.g., 330, 332, 334 of FIG. 3D) activation to stiffen the upper airway, and stylopharyngeus muscle 219 activation to open the airway. Activating pharyngeal constrictor muscle(s) may modulate or increase pharyngeal muscle tone to reduce collapsibility of the upper airway. Activating the stylopharyngeus muscle 219 may cause the pharyngeal wall(s) (e.g., posterior wall and/or side walls, with the tongue base defining the anterior portion of the oropharynx and the pharyngeal muscles do not complete encircle or form the oropharynx) to move at least one of interiorly, medial-laterally, and/or posteriorly to maintain or increase the opening of the upper airway.

In association with at least target A (and target C as well as other target locations), example sensor functions of the glossopharyngeal nerve 207 include mechanoreceptor recruitment to cause reflex opening of the airway, and chemoreceptor recruitment to prime the reflex opening activity. In some examples, the mechanoreceptor is associated with the posterior pharyngeal wall. Such mechanoreceptors may comprise a stretch receptor that sense stretching of the posterior pharyngeal wall indicative of obstructions of the upper airway. In some examples, the mechanoreceptor is a pharyngeal baroreceptor. The pharyngeal baroreceptor is believed to be in or to form part of the carotid sinus 214 and/or carotid sinus body 216. The pharyngeal baroreceptor senses changes in blood pressure. The carotid sinus body 216 is believed to form the chemoreceptor. The chemoreceptor measures pH, partial pressure CO₂, and partial pressure O₂ in the blood.

Target location B is a distal location on the stylopharyngeus nerve branch 204 of the glossopharyngeal nerve 207, which may be used to selectively activate motor functions. Target location B may include efferent fibers and not include afferent fibers. Stimulating at target location B may be used to activate the stylopharyngeus muscle 219, which may cause movement of the pharyngeal walls to maintain or increase the opening of the upper airway. As target location B does not include afferent fibers, by selectively stimulating at or near target location B, reflex activity of coughing and/or closing of the trachea may not be activated.

Target location C is a distal location on the carotid sinus branch 212 of the glossopharyngeal nerve 207, which may be used to selectively activate sensory functions. Target location C may include afferent fibers and not include efferent fibers. Stimulating at target location C may be used to enable chemoreceptor recruitment to prime and/or boost reflex activity associated with upper airway patency (e.g., reflex opening activity). For example, selective stimulation provided at or near target location C may prime a sensory pathway associated with the upper airway patency. In particular and in response to the stimulation at target location C, the carotid sinus branch 212 may be primed to receive sensory information from the chemoreceptor (e.g., the carotid sinus body 216) indicative of partial pressure CO₂, and in response, is more sensitive to the sensory information and/or responds as though the value is higher than it is. As previously described, the reflex opening activity associated with upper airway patency may include activating at least one upper airway patency-related nerve. In some examples, the upper airway patency-related nerve may comprise a hypoglossal nerve to cause at least one upper airway patency-related muscle to contract and cause protrusion of the tongue of the patient. In some examples, the at least one upper airway patency-related nerve comprises at least the hypoglossal nerve and/or the at least one upper airway patency-related muscle comprises at least protrusion muscle of a genioglossus muscle. In some examples, the upper airway patency-related nerve may comprise infrahyoid muscle-innervating nerves which may cause contraction of various infrahyoid strap muscles. In one non-limiting example, among other functions, stimulation of a sternothyroid muscle-innervating nerve may cause inferior movement of thyroid cartilage, which may in turn cause stiffening and/or increased patency of at least the oropharynx portion of the upper airway portion.

For example, in response to the chemoreceptor sensing partial pressure CO₂, the carotid sinus branch 212 (which is primed by the electrical stimulation to respond more sensitively and/or faster) provides the sensory information of partial pressure CO₂ to the brain, which then activates the hypoglossal nerve to cause at least protrusion muscles of a genioglossus muscle to contract and cause protrusion of the tongue of the patient.

Upper airway patency-related nerves include and/or refer to nerves associated with carrying sensory information and/or that stimulate muscles associated with increasing, restoring, or maintaining upper airway patency to promote respiration. Example upper airway patency-related nerves include the hypoglossal nerve, nerves innervating various infrahyoid strap muscles (sometimes herein referred to as “infrahyoid muscle-innervating nerves”), and the glossopharyngeal nerve, among other nerves.

In some examples, target C may include afferent fibers associated with other sensory pathways, such as sensory pathways that activate reflex activity associated with coughing and/or closing of the trachea. To avoid or mitigate activating reflex activity of coughing and/or closing of the trachea in response to stimulation at target location C, specifically selected afferent fibers may be stimulated, while others are not or are stimulated below a threshold level to activate the unwanted reflex activity, e.g., via steering.

Examples of stimulating the glossopharyngeal nerve 207 or other glossopharyngeal-related tissue to treat SDB are not limited to the specific target locations A, B, C of FIG. 3A, and may include different variations and/or combinations of target locations. Further, multiple target locations may be used. In some examples, the patient is stimulated on both the left side and opposite right side of the patient, such as stimulating at target location A on both the left and right sides, target location B on both the left and right sides, and/or target location C on both the left and right sides of the patient. In some examples, the stylopharyngeus muscle may be stimulated in addition to or alternatively to at least one fiber of the glossopharyngeal nerve 207, such as on the left and right sides of the patient and/or one of the left and right sides.

FIG. 3C illustrate more detail of the glossopharyngeal nerve 207 and associated structures, including originating structures and terminating structures, such as those previously described and illustrated in connection with FIG. 3A. More particularly, FIG. 3C shows the different afferent fibers 335 and efferent fibers 333 along the different branches 204, 206, 212, 331 of the glossopharyngeal nerve 207, which may be selectively stimulated to target different sensory and/or motor functions related with upper airway patency.

As described above, in some examples, selectively stimulating at a particular target location may comprise stimulating the at least one fiber while not stimulating (or stimulating below a threshold level) other fibers at that same target location of the glossopharyngeal nerve 207. The selective stimulation may be used to selectively and/or controllably cause at least one sensory function and/or at least one motor function of the glossopharyngeal nerve 207, such as those described above.

In some examples, at least one afferent fiber 335, e.g., select afferent fiber(s) 335, of a glossopharyngeal nerve 207 may be selectively stimulated, while not stimulating other fibers (e.g., efferent fibers 333 and other afferent fibers 335), such as by stimulating at target locations A and/or C. Selectively stimulating the at least one select afferent fiber 335 of the glossopharyngeal nerve 207 may induce a physiologic response and thereby maintain or increase upper airway patency.

In some examples, the physiologic response is associated with a reflex opening activity associated with upper airway patency. As previously described, one example physiologic response comprises recruiting pharyngeal mechanoreceptors (e.g., stretch receptors and/or baroreceptors) and/or recruiting chemoreceptors. In some examples, the physiologic response includes, and/or is associated with, reflex opening of the upper airway and/or priming of a sensory pathway associated with upper airway patency (and ensuing reflex opening of the airway).

For example, the physiologic response may be caused by the selective stimulation of the afferent fiber 335 (e.g., along branch 212) of the glossopharyngeal nerve 207 and is associated with the sensory pathway associated with upper airway patency. As described above, the sensory pathway may be associated with activating the at least one upper airway patency-related nerve to cause at least one upper airway patency-related muscle to contract and cause protrusion of the tongue of the patient. In some examples, the stimulation may prime the sensory pathway, such that the above-described reflex opening activity occurs more quickly and/or in response to a lower signal than without the stimulation. In some examples and referring to FIG. 3CB, the sensory pathway may be associated with a portion of the posterior pharyngeal wall (e.g., 252 in FIG. 3CB) at/in which mechanoreceptors within a zone 270 (dotted line box) are located and at/near which origin points 264A, 264B, 264C of afferent fibers (335 in FIG. 3C) of the glossopharyngeal nerve 207 begin and at which obstructions of upper airway portion 150 (e.g., within oropharynx portion 162) are sensed. Referring back to FIG. 3C, in some examples, the sensory pathway may be associated with a carotid sinus body 216 and sensing of carbon dioxide levels in the blood. In some examples and/or in addition, the sensory pathway may be associated with other mechanoreceptors.

In such examples, as previously described, selectively stimulated the at least one afferent fiber 335 of the glossopharyngeal nerve 207 may invoke a reflex opening activity of the entire array (or substantially the entire array) of upper airway patency-related muscles. For example, by stimulating the single glossopharyngeal nerve 207 (or portion thereof), via its sensory pathway(s), the therapy may invoke a comprehensive response of most or all of the upper airway patency-related muscles as part of the reflex opening activity.

In some examples, selectively stimulating comprises stimulating the at least one fiber at a target location of the glossopharyngeal nerve 207 to stimulate efferent fibers 333, e.g., select efferent fiber(s) 333, of the glossopharyngeal nerve 207 while not stimulating afferent fibers 335 of the glossopharyngeal nerve, such as by stimulating efferent fibers 333 at the target locations A and/or B illustrated in connection with FIG. 3A. Stimulating the select efferent fiber(s) 333 may activate upper airway patency-related muscle, such as the stylopharyngeus muscle 219 and/or at least one pharyngeal constrictor muscle, as illustrated by 330, 332, 334 of FIG. 3D.

In some examples, target location B may be used to selectively stimulate at least one efferent fiber 333 (e.g., along branch 204) of the glossopharyngeal nerve 207 and, in response, to activate the stylopharyngeus muscle 219 and without activating at least one of coughing and trachea closure. As the target location B includes efferent fibers 333 and not afferent fibers 335, as previously described, stimulating at target location B may not stimulate the afferent fibers 335 of the glossopharyngeal nerve 207. Activating the stylopharyngeus muscle 219 may increase opening of the upper airway.

In some examples, target A may be used to selectively stimulate at least one efferent fiber 333 of the glossopharyngeal nerve 207 and, in response, to activate at least one pharyngeal constrictor muscle, as illustrated by 330, 332, 334 of FIG. 3D. Activating the at least one pharyngeal constrictor muscle may stiffen the upper airway (e.g., increases pharyngeal muscle tone) and, in response, reduce collapsibility of the upper airway. In some examples, the stimulation may comprise applying electrical stimulation, via at least one stimulation element, to the at least one efferent fiber 333 steadily during a treatment period (or at least a selected portion of a treatment period) and below a threshold level, as further described herein. In some such examples, this steady stimulation may sometimes be referred to as continuous stimulation in the sense that is maintained throughout most or all of a treatment period (or selected portion thereof) without substantial interruption.

In some examples, both the stylopharyngeus muscle 219 and the at least one pharyngeal constrictor muscle (e.g., 330, 332, 334) may be activated, such as by stimulating at target location A.

In some examples, multiple fibers of the glossopharyngeal nerve 207 are stimulated at the same time. For example, at least one afferent fiber 335 of the glossopharyngeal nerve 207 at a proximal location, and at least one efferent fiber 333 of the glossopharyngeal nerve 207 at the proximal location may be stimulated, such as by using target location A. In such examples, both sensory and motor functions of the glossopharyngeal nerve 207 are activated, such as inducing a physiologic response comprising a motor function and a sensory function of glossopharyngeal nerve 207.

In some examples, the stimulation of the glossopharyngeal-related tissue (e.g., selective stimulating of the at least one fiber of the glossopharyngeal nerve 207) is associated with timing and/or an amount of stimulation applied. For example, the stimulating may comprise applying the selective stimulation based on respiration information, applying a plurality of electrical stimulations at a duty cycle, and/or applying electrical stimulation (e.g., steady) below a threshold level, among other variations. In some examples, the stimulation may be response to at least one detected SDB event.

In some examples, as described above, when applying electrical stimulation to at least one afferent fiber 335 (e.g., at target locations A or C) and/or to at least one efferent fiber 333 to cause contraction of at least one pharyngeal constrictor muscle (e.g., 330, 332, 334 of FIG. 3D at target location A), the electrical stimulation may be applied without regard to detection of SBD event(s) and/or without regard to timing with respiration. For example, the electrical stimulation may include applying electrical stimulation during a treatment period and below a threshold level (e.g., steady) and/or applying a plurality of electrical stimulations at a duty cycle while the patient is in a sleep state. Such stimulation may cause the muscle to exhibit tone without fully contracting.

In some examples, the selective stimulating of the at least one fiber (e.g., efferent fiber 333) of the glossopharyngeal nerve 207 is associated with timing the stimulation with respiration of the patient. For example, the electrical stimulation may be applied in response to detection of an SDB event and/or timed with respiration, such as when stimulating at target location B and/or at target location A to activate the stylopharyngeus muscle 219. As previously described, activating the stylopharyngeus muscle 219 may cause the pharyngeal wall to move, thereby opening the upper airway, and which may be useful to be timed with the inspiration phase of respiration. In some such examples, respiration information indicative of a respiratory cycle may be sensed using a sensor, and the stimulation is selectively applied based on the respiratory cycle. Example respiration information includes sensed respiratory phase information, which comprises at least one of an inspiratory phase and an expiratory phase, respiratory rate, among other information.

As noted above, in some examples, the stimulation is caused in response to at least one SDB event or the stimulation may be timed relative to at least some other parameter. At least some example parameters include respiration information, cardiac information, body position, and/or sleep state, among other sensed physiologic information. Other example parameters may comprise an upper airway obstruction or otherwise detectable parameter indicative of at least one SDB event. For example, at least one parameter may be sensed using at least one sensor, and based on the at least one parameter, an SDB event rate (e.g., apnea events per hour) is identified and the at least one fiber of the glossopharyngeal nerve 207 or other glossopharyngeal-related tissue is stimulated. In some examples, stimulating is applied by modulating the stimulation based on at least one of the respiration information, body position, a sleep state of the patient, and/or timing the stimulation relative to the respiration information of the patient.

FIG. 3CA is a close-up view of target locations A, B, and C of FIG. 3C. As shown by FIG. 3CA, target location A includes efferent fibers 333 and afferent fibers 335 of the glossopharyngeal nerve 207. Target location B includes efferent fibers 333 of the stylopharyngeus nerve branch 204 of the glossopharyngeal nerve 207. And, target location C includes afferent fibers 335 of the carotid sinus branch 212 of the glossopharyngeal nerve 207.

FIG. 3CB illustrates example mechanoreceptors. As shown by FIG. 3CB, target location D associated with zone 270 includes origin points 264A, 264B, 264C of afferent fibers (e.g., 335 of FIG. 3C) of the glossopharyngeal nerve 207. The afferent fibers associated with origin points 264A, 264B, 264C extend from mechanoreceptors that are located at the posterior pharyngeal wall 252 to the brain and carry the sensory information indicative of changes in stretch at the posterior pharyngeal wall 252. More particularly, at origin points 264A, 264B, 264C of afferent fibers (335), sensory information indicative of obstructions of upper airway portion 150 (e.g., within oropharynx portion 162) is sensed and provided to the brain. In response to the sensory information, the brain sends signals to at least one upper airway patency-related tissue to cause activation of at least one upper airway patency-related muscles, such as activating an array of upper airway patency-related muscles to provide a more comprehensive physiological response as compared to stimulating a single nerve and/or muscle (e.g., hypoglossal nerve or genioglossus muscle).

In any of the above described example, accessing the at least one fiber, such as an afferent fiber, may be difficult. For example, delivering a stimulation electrode arrangement at any of the target locations may be difficult. In some examples, the stimulation location may be at more proximal portions of the glossopharyngeal nerve 207 than the illustrated target locations A, B, C, and D, which may be more practical for accessing and delivering stimulation electrode arrangements to, and the at least one fiber may be selectively stimulated (e.g., stimulate the afferent and not efferent fibers). In some examples, the stimulation location may be intermediate locations, such as between origin points 264A, 264B and a more proximal location. In such some examples, a selective stimulation protocol (and stimulation electrode arrangement) may be used to distinguish the intended fibers (e.g., afferent fibers) from others, such as distinguishing the afferent fibers from pharyngeal wall/mechanoreceptors from the afferent fibers originating from or near carotid sinus (chemoreceptor, baroreceptor) and/or efferent fibers.

FIG. 3D is side view of a neck region of a patient and different upper airway patency-related muscle which may be innervated by stimulating at least one fiber of the glossopharyngeal nerve, such as at the target locations A, B, C of FIGS. 3A and 3C. More particularly, FIG. 3D illustrates the stylopharyngeus muscle 219 and the pharyngeal constrictor muscles 330, 332, 334.

In some examples, stimulating the at least one fiber of the glossopharyngeal nerve, such as selectively stimulating respective efferent fiber(s) at target location A or target location B of FIG. 3A, may activate at least one stylopharyngeus muscle 219. As described above, in some examples, both the left and right sides of the patient may be stimulated at the glossopharyngeal nerve, such that the stylopharyngeus muscles 219 are activated on the left and right sides of the patient. Activating the at least one stylopharyngeus muscle 219 may increase the opening of the upper airway by causing the pharyngeal walls to move.

In some examples, the at least one stylopharyngeus muscle 219 may be stimulated to open the upper airway, such as to increase or maintain patency of at least the oropharynx portion of the upper airway. For example, the at least one stylopharyngeus muscle 219 may be directly stimulated using at least one stimulation element, in addition to or alternatively to the stimulation of the at least one fiber of the glossopharyngeal nerve. At least some patients may respond better to direct stimulation of the at least one stylopharyngeus muscle 219 to promote upper airway patency as compared to stimulating the glossopharyngeal nerve. The at least one fiber of the glossopharyngeal nerve may be easier to gain access to and/or to stimulate than the stylopharyngeus muscle 219, such as for implantation purposes.

In some examples, stimulating the at least one fiber of the glossopharyngeal nerve, such as selectively stimulating respective efferent fiber(s) at target location A of FIG. 3A, may activate least one pharyngeal constrictor muscle 330, 332, 334. In some examples, the middle constrictor 330 and inferior constrictor 334 are activated. In some examples, the superior constrictor 332, the middle constrictor 330, and inferior constrictor 334 are activated. In some examples, the middle constrictor 330 is activated. As described above, in some examples, both the left and right sides of the patient may be stimulated by the glossopharyngeal nerve, such that the at least one pharyngeal constrictor muscle 330, 332, 334 is activated on the left and right sides of the patient. Activating the at least one pharyngeal constrictor muscle 330, 332, 334 may stiffen the upper airway (e.g., increases pharyngeal muscle tone) and reduce collapsibility of the upper airway.

In some examples, the selective stimulation to activate the at least one constrictor muscle 330, 332, 334 may include applying at least one electrical stimulation that is below a threshold (e.g., energy level) to the at least one efferent fiber to activate the at least one constrictor muscle 330, 332, 334. By applying the electrical stimulation below a threshold level, reflex activity associated with coughing and/or closing of the trachea may be avoid and/or mitigated. In some examples, the stimulation includes applying a plurality of electrical stimulations at a duty cycle, each below the threshold, or applying a (steady) electrical stimulation below the threshold during a treatment period, sometimes referred to as a tonic low-level stimulation.

FIGS. 4A-4B illustrate example timing diagrams for a CO₂ sensor of the body. As described above, the chemoreceptor of the carotid sinus body (216 of FIGS. 3A-3C) may act as a CO₂ sensor of the body. By stimulating at least one afferent fiber of the glossopharyngeal nerve, such as an afferent fiber of the carotid sinus branch 212 of FIG. 3A, the sensory pathway associated with the CO₂ sensor of the body may be gated or primed. As such, stimulating the afferent fiber of the glossopharyngeal nerve may be used to mediate activation of opening of the airway and/or for increasing or maintaining upper airway patency.

The timing diagrams 441, 443 of FIGS. 4A-4B respectively show stable partial pressure CO₂ (diagram 441) and partial pressure CO₂ above a threshold (diagram 443) for a patient. In the timing diagram 441, the partial pressure CO₂ (PₑₜCO₂) is stable at circles A and B, with relative stability of the genioglossus muscle shown at circle C, thus showing that when the partial pressure CO₂ remains below a threshold, the genioglossus muscle is not activated. In the timing diagram 443, the PₑₜCO₂ is increasing from circle D to circle E, going from 32 mmHg to 40 mmHG. As the relatively high (40 mmHg0) PₑₜCO₂ is reached, the brain recognizes there is too much CO₂ (via sensory signal carried from the carotid sinus body through the glossopharyngeal nerve), which causes a physiologic response or reflex opening activity including activating nerves to open the upper airway. For example, among other nerves and muscles responsible for increasing and/or maintaining upper airway patency and which become activated via the reflex opening activity, via its innervation of the genioglossus muscle and upon its activation as part of the reflex opening activity, the hypoglossal nerve causes the genioglossus muscle to contract, as shown by circle F, resulting in the tongue protrusion and opening of the upper airway.

In some examples, the sensory pathway which causes the brain to recognize the CO₂ levels is too high, and causes the physiologic response, is primed by the selective stimulation of the at least one afferent fiber of the glossopharyngeal nerves, such as described in connection with the method 10 of FIG. 1 and/or by stimulating at target location C illustrated by FIGS. 3A and 3C. Examples are not so limited and may include stimulating efferent fibers, stimulating the stylopharyngeus muscle directly, and/or other sensory pathways.

In such examples, as previously described, selectively stimulated the at least one afferent fiber of the glossopharyngeal nerve may invoke a reflex opening activity of the entire array (or substantially the entire array) of upper airway patency-related muscles. For example, by stimulating the single glossopharyngeal nerve (or portion thereof), via its sensory pathway, the therapy may invoke a comprehensive response of most or all of the upper airway patency-related muscles as part of the reflex opening activity.

FIGS. 5A-5F are diagrams schematically representing deployment of example stimulation elements and example implantable medical devices (IMDs). Example IMDs may be used to stimulate nerves and/or muscles, such as to stimulate at least one fiber of a glossopharyngeal nerve in accordance with any of the methods of at least FIGS. 1 and 9A-9C, as well as the various variations as described in connection with at least FIGS. 2A-4B, 6A-8, 10-11.

More specifically, FIG. 5A is a block diagram schematically representing an example IMD 1280. The IMD 1280 may include a stimulation element. The stimulation element may include an IPG assembly 1281 and at least one stimulation lead 1255. The IPG assembly 1281 may include a housing 1283 containing circuitry 1286 and a power source 1288 (e.g., battery), and an interface block or header-connector 1284 carried or formed by the housing 1283. The housing 1283 is configured to render the IPG assembly 1281 appropriate for implantation into a human body, and may incorporate biocompatible materials and hermetic seal(s). The circuitry 1286 may be implemented, at least in part, via a control portion 1300 (and related functions, portions, elements, engines, parameters, etc.) such as described later in connection with at least FIGS. 6A-15.

In some examples, the stimulation lead 1255 includes a lead body 1250 with a distally located stimulation electrode arrangement 1260. At an opposite end of the lead body 1250, the stimulation lead 1255 includes a proximally located plug-in connector 1282 which is configured to be removably connectable to the interface block 1284. The interface block 1284 may include or provide a stimulation port sized and shaped to receive the plug-in connector 1284.

In general terms, the stimulation electrode arrangement 1260 may optionally be a cuff electrode, and/or may include some non-conductive structures biased to (or otherwise configurable to) releasable secure the stimulation electrode arrangement 1260 about a target nerve or other target tissue, e.g., the glossopharyngeal nerve. Other formats are also acceptable. In some examples, the stimulation electrode arrangement 1260 may include an array of contact electrodes to deliver a stimulation signal to a target nerve. Examples are not limited to cuffs and may include stimulation elements having a stimulation electrode arrangement 1260 in different types of arrangements and/or for different targets, such as an alternating current (AC) target, a paddle, and an axial arrangement, among others. The stimulation electrode arrangement(s) may contact a target tissue and/or otherwise be in stimulating relation to the target tissue in a non-contact manner.

In some examples, the lead body 1250 comprises a generally flexible elongate member having sufficient resilience to enable advancing and maneuvering the lead body 1250 subcutaneously to place the stimulation electrode 1260 at a desired location adjacent target tissue, such as an upper airway patency-related nerve or muscle. In some examples, such as in the case of OSA, the nerves may include (but are not limited to) the glossopharyngeal nerve and the muscles may include (but are not limited to) the stylopharyngeus muscle and/or pharyngeal constrictor muscles. In some examples, the nerves may include (but are not limited to) the hypoglossal nerve and associated muscles responsible for causing movement of the tongue and related musculature to restore airway patency. Other nerves suitable for stimulation may comprise an infrahyoid muscle-innervating nerve to cause contraction of at least some infrahyoid strap muscles to increase and/or maintain upper airway patency, such as in the oropharynx portion. In some examples, at least one infrahyoid muscle-innervating nerve may comprise an ansa cervicalis-related nerve.

In some examples, lead body 1250 may have a length sufficient to extend from the IPG assembly 1281 implanted in one body location (e.g., pectoral) and to the target stimulation location (e.g., head, neck). Upon generation via the circuitry 1286, a stimulation signal is selectively transmitted to the interface block 1284 for delivery via the stimulation lead 1255 to, such as at least one fiber of the glossopharyngeal nerve.

It will be understood that the interface block 1284 is representative of many different kinds and styles of electrical (and mechanical) connection between the housing of the IPG assembly 1281 and the lead 1255 with such connections having a size, shape, location, etc., which may differ from the interface block 1284 shown in FIG. 5A.

In some examples, the IMD 1280 further comprises at least one implantable sensor 1285. The at least one implantable sensor 1285 may be connected to the IMD 1280 in various fashions, such as being coupled to the interface block 1284, being carried by (or within) the IPG assembly 1281, and/or wirelessly communicating with the IPG assembly 1281. More specifically, the at least one implantable sensor 1285 may be connected in various orientations as described within U.S. Patent Publication No. 2021/0268279, published on Sep. 2, 2021, and entitled “SYSTEMS AND METHODS FOR OPERATING AN IMPLANTABLE MEDICAL DEVICE BASED UPON SENSED POSTURE INFORMATION”, the entire teachings of which is incorporated herein by reference in its entirety, including the determining or designating a posture of the patient based on data from the acceleration sensor. Although the above examples describe an IMD 1280 having a stimulation lead 1255, examples are not so limited and example IMDs may additionally or alternatively include a lead used for sensing.

It will be understood that the example IMDs in FIGS. 5A-5F are not limited to the example sensors described in association with FIGS. 5A-5F but may comprise at least one of the different sensor modalities, placements, etc. described later in association with at least FIGS. 9A-9C and 11 and/or generally throughout the present disclosure.

In some examples, the at least one implantable sensor 1285 may be wirelessly connected to the IMD 1263. In such examples, the interface block 1284 need not provide a sense port for the at least one implantable sensor 1285 or the sense port may be used for a second sensor (not shown). In some examples, the circuitry 1286 of the IPG assembly 1281 and circuitry of the at least one implantable sensor 1285 communicate via a wireless communication pathway according to known wireless protocols, such as Bluetooth, near-field communication (NFC), Medical Implant Communication Service (MICS), 802.11, etc. with each of the circuitry 1286 and the at least one implantable sensor 1285 including corresponding components for implementing the wireless communication pathway. In some examples, a similar wireless pathway is implemented to communicate with devices external to the patient's body for at least partially controlling the at least one implantable sensor 1285 and/or the IPG assembly 1281, to communicate with other devices (e.g., other sensors) internally within the patient's body, or to communicate with other sensors external to the patient's body.

FIG. 5B is diagram including a front view schematically representing deployment 1200 of an example IMD 1262 which includes at least one stimulation element. In some examples, the stimulation element comprises an IPG 1233 and at least one stimulation electrode arrangement 1201. As shown in FIG. 5B, in some examples the IPG 1233 may be chronically implanted in a pectoral region 1232 of a patient and the stimulation electrode arrangement 1201A may be chronically implanted at or near a head region 1203 of the patient and/or superior to the neck region 1205 of the patient. The at least one stimulation electrode arrangement 1201 may be implanted near or coupled to the glossopharyngeal nerve 1207 (or other glossopharyngeal-related tissue), such as illustrated by the target locations A, B, C of FIGS. 3A and 3C. The stimulation electrode arrangement 1201 may include an implementation of, and/or including at least some of substantially the same elements and features as, the example stimulation elements and/or electrode arrangements previously described in connection with at least FIGS. 1-5A or as later described in connection with at least FIGS. 10-11. The common elements and features are not repeated for ease of reference. Among other features, it will be understood that in some examples a body of a lead 1255 (FIG. 5A) supports the stimulation electrode arrangement 1201, while extending between the IPG 1233 and the stimulation electrode arrangement 1201. Moreover, in some examples, the IPG 1233 may be formed on a smaller scale (e.g., microstimulator) and/or different shape to be amenable for implantation in a head and/or neck regions 1203, 1205 instead of pectoral region 1232.

FIG. 5C is a diagram including a front view schematically representing deployment 1210 of an example IMD 1270 which includes a stimulation element comprising an IPG 1233 and at least one stimulation electrode arrangement 1201. In some examples, IMD 1270 includes an implementation of, and/or at least some of substantially the same features and attributes as, the IMD 1262 as previously described in connection with at least FIG. 5B, and the IPG 1233 may be implanted in a pectoral region 1232 and/or include a sensor, as previously described. The common elements and features are not repeated for ease of reference.

In some examples, the IMD 1270 comprises a lead 1272 including a lead body 1278 (e.g., 1250/1255 in FIG. 5A) for chronic implantation (e.g., subcutaneously via tunneling or other techniques) and to extend from the IPG 1233 to a position adjacent to the at least one nerve 1207 (or other tissue) and with at least one stimulation electrode arrangement 1201 on an opposite end of the IPG 1233. The stimulation electrode arrangement 1201 may engage the nerve 1207 in a head region 1203 for stimulating the nerve to treat a physiologic condition, such as SDB.

In some such examples, multiple nerves may be targeted for stimulation, such as illustrated by FIG. 5F, and separate stimulation leads may be provided or a single stimulation lead may be provided but with a bifurcated distal portion with each separate distal portion extending to a respective one of the multiple nerves. For example, and referring to FIG. 5F, a stimulation lead, on which the least one stimulation electrode of the at least one stimulation electrode arrangement 1201A, 1201B, 1201C is supported, may be implanted in a position extending between the IPG 1233 and a stimulating relation to the at least one nerve 1207, 1209, 1211. Further details regarding applying stimulation to multiple target tissues is described later in association with at least FIGS. 5E-5F and 10-12H.

FIG. 5D is a diagram including a front view schematically representing deployment 1215 of an IMD 1249A comprising at least some of substantially the same features and attributes as the IMD 1262 in FIG. 5B, except with the IPG 1233 implemented as a microstimulator 1249B. In some examples, the microstimulator 1249B may be chronically implanted (e.g., percutaneously, subcutaneously, transvenously, etc.) in a head region and/or neck region 1203, 1205 as shown in FIG. 5D, or in a pectoral region 1232. In some examples, as part of the IMD 1249A, the microstimulator 1249B may be in wired or wireless communication with the at least one stimulation electrode of stimulation electrode arrangement 1201. In some examples, as part of the IMD 1249A, the microstimulator 1249B may incorporate sensor or be in wireless or wired communication with a sensor located separately from a body of the microstimulator 1249B. When wireless communication is employed for sensing and/or stimulation, the microstimulator 1249B may be referred to as leadless IMD for purposes of sensing and/or stimulation. In some examples, the microstimulator 1249B may be in close proximity to a target nerve 1207.

In some examples, the microstimulator 1249B (and associated elements) may comprise at least some of substantially the same features and attributes as described and illustrated in U.S. Patent Publication No. 2020/0254249, published on Aug. 13, 2020, and entitled “MICROSTIMULATION SLEEP DISORDERED BREATHING (SDB) THERAPY DEVICE”, the entire teachings of which is incorporated herein by reference in its entirety.

While FIGS. 5A-5D illustrate a single stimulation electrode arrangement, in some examples multiple stimulation electrode arrangements may be implanted in the patient. For example, and as shown in FIGS. 5E-5F, the at least one stimulation element comprises a first electrode arrangement 1201A and a second electrode arrangement 1201B, or more. In some such examples, applying the stimulation comprises applying the stimulation on both a first portion and a second portion, e.g., a right side 1221 and opposite left side 1223 of FIG. 5E or different areas of the head 1203 or neck 1205 of a body of the patient via the first stimulation electrode arrangement 1201A and the second stimulation electrode arrangement 1201B.

FIG. 5E is a diagram including a front view schematically representing deployment 1217 of at least two stimulation electrode arrangements 1201A, 1201B. The electrode arrangements 1201A, 1201B may include an implementation of, and/or at least some of substantially the same elements and features as, the stimulation element and/or electrode arrangements previously described in connection with the examples of at least FIGS. 1-5D. Further, in some examples, a PG, which is implanted or wearable, may be used to provide electrical signals to the stimulation electrode arrangements 1201A, 1201B, as previously described in connection with at least FIGS. 1-5D.

As shown by FIG. 5E, each stimulation electrode arrangements 1201A, 1201B may be chronically implanted at or near a head region 1203 of the patient and/or superior to the neck region 1205 of the patient, with the first stimulation electrode arrangement 1201A being implanted on a right side 1221 of the patient and the second stimulation electrode arrangement 1201B being implanted on an opposite left side 1223 of the patient. Each stimulation electrode arrangement 1201A, 1201B may be implanted near or coupled to the glossopharyngeal nerves 1207A, 1207B on the right and left sides 1221, 1223 of the head region 1203 of the patient, such as illustrated by the target locations A, B, C of FIG. 3A. However, examples are not so limited and, in some examples, target location C may be in the neck region 1205 of the patient.

In some examples, additional stimulation electrode arrangements and/or stimulation elements may be implanted, such as to target multiple target locations of the glossopharyngeal nerves 1207A, 1207B and/or to target other upper airway patency-related tissue, including but not limited to the stylopharyngeus muscle, the hypoglossal nerve, the genioglossus muscle, at least some infrahyoid strap muscles and those nerves innervating the infrahyoid strap muscles, among other types of nerve and muscle tissue.

FIG. 5F is a diagram including a front view schematically representing deployment 1219 of an IMD 1263 (and/or example method). In some examples, the IMD 1263 may include stimulation element including an IPG 1233 implanted in a pectoral region 1232 of the patient's body and including at least one sensor 1268A. In some examples, IMD 1263 comprises an implementation of, and/or at least some of substantially the same features and attributes as, any of the IMDs as previously described in connection with any of FIGS. 5A-5E. Accordingly, in some examples, sensor 1268A may comprise at least an acceleration sensor having at least some of substantially the same features and attributes as further described later in connection with at least FIGS. 9A-9C. Via such example sensing arrangements, the IMD 1263 may identify the body posture, SDB obstruction or event(s), respiration information, cardiac information, sleep state, and/or other SDB-parameters. Further, in some examples, any of the IMDs and/or non-implanted devices described herein may include sensor 1268A and/or any of the sensors used to detect parameters, as further described in connection with FIGS. 9A-9C.

As further shown in FIG. 5F, the IMD 1263 comprises at least one stimulation element including at least one stimulation electrode arrangement 1201A, 1201B, 1201C for chronic implantation (e.g., subcutaneously via tunneling or other techniques) at a position adjacent a nerve (e.g., glossopharyngeal nerve 1207, hypoglossal nerve 1209, infrahyoid strap muscle-innervating nerve 1211) and/or a muscle. The stimulation electrode arrangements 1201A, 1201B, 1201C may comprise electrodes to engage the nerve, e.g., 1207, 1209, 1211 in a head region and/or neck region 1203, 1205 for stimulating the nerve to treat a physiologic condition, such as SDB. The IMD 1263 (and any of the IMDs illustrated by FIGS. 5A-5F) may comprise a control portion (e.g., circuitry, power element, etc.) to support control and operation of both the sensor 1268A and the stimulation electrode arrangements 1201A, 1201B, 1201C (via lead(s), not shown, or wirelessly). In some examples, such control, operation, etc., may be implemented, at least in part, via a control portion 1300 (and related functions, portions, elements, engines, parameters, etc.) such as described later in connection with at least FIGS. 6A-11.

With regard to the various examples, delivering stimulation to at least one fiber of the glossopharyngeal nerve 1207 (or other glossopharyngeal-related tissue), and optionally, an additional upper airway patency nerve (e.g., a hypoglossal nerve 1209 and/or infrahyoid muscle-innervating nerve 1211) via the at least one stimulation electrode arrangement 1201A, 1201B, 1201C may activate or prime a sensory pathway associated with upper airway patency and/or cause contraction of upper airway patency-related muscles, which may cause or maintain opening of the upper airway to prevent and/or treat OSA, as previously described in connection with FIGS. 1-5E and as further described later in associated with at least FIGS. 10-15.

In some examples, the IPG 1233 may be in wired or wireless communication with stimulation electrode arrangement(s) 1201A, 1201B, 1201C and/or with a sensor 1268B, 1268C located separately from a body of the IPG 1233. When wireless communication is employed for sensing and/or stimulation, the IPG 1233 may be referred to as leadless IMD, as previously described. In some examples, the IPG 1233 may be in close proximity to a target nerve 1207.

As further shown in the diagram of FIG. 5F, in some examples, the IMD 1263 may be implemented with additional sensors 1268B, 1268C to sense bio-impedance, ECG, and/or other sensing modalities, from which additional physiologic data such as, but not limited to, further respiration information may be determined. In some examples, one or both of the sensors 1268B, 1268C may comprise sensing electrodes. In some examples, stimulation electrode arrangement(s) 1201A, 1201B, 1201C also may act, in some examples, as a sensing electrode. In some examples, at least a portion of housing of the IPG 1233 also may comprise a sensor 1268A or at least an electrically conductive portion (e.g., electrode) to work in cooperation with at least some of the above-mentioned sensing electrodes to implement at least some sensing arrangements to sense bio-impedance, ECG, etc., as described above.

As implicated by the above description, the IMD of the examples of the present disclosure includes a controller, control unit, or control portion that prompts, controls, tracks, etc., performance of designated actions.

FIG. 6A is a block diagram schematically representing an example control portion. In some examples, the control portion 1300 includes a controller (e.g., processor) 1302 and a memory 1310. In some examples, the control portion 1300 provides one example implementation of a control portion forming a part of, implementing, and/or managing any one of devices, assemblies, circuitry, managers, engines, functions, parameters, respiration determination elements, stimulation elements, pulse generators, sensors, electrodes, modules, and/or methods, as represented throughout the present disclosure in association with FIGS. 1-5F and 11-15.

The control portion 1300 may include circuitry components and wiring appropriate for generating desired stimulation signals (e.g., converting energy provided by the power source into a desired stimulation signal), for example in the form of the care engine 1311. In some examples, the control portion 1300 may include telemetry components for communication with external devices. For example, the control portion 1300 may include a transmitter that transforms electrical power into a signal associated with transmitted data packets, a receiver that transforms a signal into electrical power, a combination transmitter/receiver (or transceiver), an antenna (e.g., an inductive telemetry antenna), etc.

In general terms, the controller 1302 of the control portion 1300 comprises an electronics assembly 1304 (e.g., at least one processor, microprocessor, integrated circuits, and logic, etc.) and associated memories or storage devices. The controller 1302 is electrically couplable to, and in communication with, the memory 1310 to generate control signals to direct operation of at least some the devices, assemblies, circuitry, managers, modules, engines, functions, parameters, respiration determination elements, stimulation elements, PGs, sensors, electrodes, and/or methods, as represented throughout the present disclosure. In some examples, these generated control signals include, but are not limited to, employing the stimulation of upper airway patency-related tissues and associated functions which facilitate such stimulation. In some such examples, the upper airway patency-related tissue may comprise at least one fiber of the glossopharyngeal nerve, among other nerves and/or muscles. The control signals may be a software program stored on the memory 1310 (which may be stored on another storage device and loaded onto the memory 1310), and executed by the electronics assembly 1304. In some such examples, the control signals may at least identify stimulation to apply, and optionally, information regarding respiration, upper airway obstruction (which may come from respiration information in some examples), cardiac body position, sleep state, and/or other physiologic (or other) phenomenon. In addition, and in some examples, these generated control signals include, but are not limited to, employing the care engine 1311 stored in the memory 1310 to at least manage care provided to the patient, for example therapy for SDB (or other therapies, such as cardiac), with such care, in at least some examples, including stimulating the glossopharyngeal nerve or other glossopharyngeal-related tissue.

In response to or based upon commands received via a user interface (e.g., user interface 1420 in FIG. 7), sensor signals, and/or via machine readable instructions, controller 1302 generates control signals as described above in accordance with examples of the present disclosure. In some examples, controller 1302 is embodied in a general purpose computing device while in some examples, controller 1302 is incorporated into or associated with at least some of the sensors, respiration determination elements, stimulation elements, PGs, devices, user interfaces, instructions, information, engines, functions, actions, and/or method, etc., as described herein.

For purposes of this application, in reference to the controller 1302, the term “processor” shall mean a presently developed or future developed processor (or processing resources) that executes machine readable instructions contained in a memory. In some examples, execution of the machine readable instructions, such as those provided via memory 1310 of control portion 1300 cause the processor to perform the above-identified actions, such as operating controller 1302 to implement the sensing, monitoring, identifying the upper airway obstruction, stimulating, and/or treatment, etc. as generally described in (or consistent with) at least some examples of the present disclosure. The machine readable instructions may be loaded in a random access memory (RAM) for execution by the processor from their stored location in a read only memory (ROM), a mass storage device, or some other persistent storage (e.g., non-transitory tangible medium or non-volatile tangible medium), as represented by memory 1310. In some examples, the machine readable instructions may comprise a sequence of instructions, or the like. In some examples, memory 1310 comprises a computer readable tangible medium providing non-volatile storage of the machine readable instructions executable by a processor of controller 1302. In some examples, the computer readable tangible medium may sometimes be referred to as, and/or comprise at least a portion of, a computer program product. In some examples, hard wired circuitry may be used in place of or in combination with machine readable instructions to implement the functions described. For example, controller 1302 may be embodied as part of at least one application-specific integrated circuit (ASIC), at least one field-programmable gate array (FPGA), and/or the like. In some examples, the controller 1302 is not limited to any specific combination of hardware circuitry and machine readable instructions, nor limited to any particular source for the machine readable instructions executed by the controller 1302.

In some examples, control portion 1300 may be entirely implemented within or by a stand-alone device.

In some examples, the control portion 1300 may be partially implemented in one of the sensors, respiration determination elements, monitoring devices, stimulation devices, etc., and partially implemented in a computing resource (e.g., at least one external resource) separate from, and independent of, the IMD (or portions thereof or a non-implanted medical device) but in communication with the IMD (or portions thereof). For instance, in some examples control portion 1300 may be implemented via a server accessible via the cloud and/or other network pathways. In some examples, the control portion 1300 may be distributed or apportioned among multiple devices or resources such as among a server, an apnea treatment device (or portion thereof), and/or a user interface.

In some examples, control portion 1300 includes, and/or is in communication with, a user interface 1420 as shown in FIG. 7.

FIG. 6B is a diagram schematically illustrating example arrangements of a control portion by which the control portion 1300 (FIG. 6A) may be implemented. In some examples, control portion 1328 is entirely implemented within or by an IPG (or non-implanted PG) 1325, which has at least some of substantially the same features and attributes as an IPG, as previously described throughout the present disclosure. In some examples, control portion 1328 is entirely implemented within or by a remote control 1330 (e.g., a programmer) external to the patient's body, such as a patient control 1332 and/or a physician control 1334. In some examples, the control portion 1328 is partially implemented in the IPG 1325 and partially implemented in the remote control 1330 (at least one of patient control 1332 and physician control 1334).

FIG. 7 is a block diagram schematically representing a user interface. In some examples, user interface 1420 forms part of and/or is accessible via a device external to the patient and by which the medical device and/or other IMD may be at least partially controlled and/or monitored. The external device which hosts user interface 1420 may be a patient remote (e.g., 1332 in FIG. 6B), a physician remote (e.g., 1334 in FIG. 6B) and/or a clinician portal. In some examples, user interface 1420 comprises a user interface or other display that provides for the simultaneous display, activation, and/or operation of at least some of the sensors, respiration determination elements, stimulation elements, PGs, devices, user interfaces, instructions, information, modules, engines, functions, actions, and/or method, etc., as described in connection with FIGS. 1-6B. In some examples, at least some portions or aspects of the user interface 1420 are provided via a graphical user interface (GUI), and may comprise a display 1424 and input 1422.

FIG. 8 is a block diagram 1500 which schematically represents some example implementations by which an implantable device may communicate wirelessly with external circuitry outside the patient. As described above, the controller and/or control portion of at least one PG 1510 illustrated in FIG. 8 may be implemented by components of the PG 1510, components of external devices (e.g., mobile device 1520, patient remote control 1540, a clinician programmer 1550, and a patient management tool 1560), and various combinations thereof. The PG 1510 may include an IPG and/or other medical device, as described above, and/or may comprise at least some aspects of a medical device implemented as further described later in association with at least FIGS. 10-11. As shown in FIG. 8, in some examples, the PG 1510 may communicate with at least one of patient application 1530 on a mobile device 1520, a patient remote control 1540, a clinician programmer 1550, and a patient management tool 1560. The patient management tool 1560 may be implemented via a cloud-based portal 1562, the patient application 1530, and/or the patient remote control 1540. Among other types of data, these communication arrangements enable the PG 1510 to communicate, display, manage, etc., the therapy provided, as well as to allow for adjustment to the various elements, portions, etc. of the example devices and methods if and where desired. In some examples, the various forms of therapy provided may be displayed to a patient and/or clinician via one of the above-described external devices.

FIGS. 9A-9B are diagrams schematically representing an example method comprising selectively applying different care, while FIG. 9C is a block diagram schematically representing an example sensing portion. The diagrams may comprise part of, and/or are example implementations of, method 10 of FIG. 1 and/or any of the variations as described through the present disclosure and in connection with FIGS. 2A-15. Furthermore, any of the apparatuses and/or devices illustrated and described in connection with FIGS. 2A-5F may be used to implement the method illustrated by FIG. 9A, may include the control portion of FIG. 9B, the sensing portion of FIG. 9C, and/or the stimulation portion of a care engine as further described in associated with at least FIGS. 10-11.

FIG. 9A is a flow diagram illustrating an example method 900 for selectively applying care. In some examples, method 900 may comprise an example implementation of, and/or be performed via at least some of substantially the same features and attributes as, the example devices, assemblies, circuitry, managers, modules, engines, functions, parameters, respiration determination elements, stimulation elements, PGs, sensors, electrodes, and/or methods described in association with FIGS. 1-8, 9B, and 10-11. In some examples, method 900 may be performed via at least some devices, assemblies, circuitry, managers, modules, engines, functions, parameters, respiration determination elements, stimulation elements, PGs, sensors, electrodes, and/or methods other than those described in association with FIGS. 1-15.

In some examples, the method 901 of applying care is optionally based on at least one sensed parameter. The at least one parameter may be sensed via at least one sensor. As further illustrated by FIG. 9B, the at least one sensor may be integrated with (or form part of) the PG and/or may be external to the PG such as an implanted sensor, a wearable sensor, or other types of sensors external to the patient, external therapy components, etc., at least some aspects of which are further described later in association with example arrangement 3100 of FIG. 11.

In some examples, the method 900 of FIG. 9A may be performed by a control portion implemented as the control portion 1300 of FIG. 6A, such as a control portion of the pulse generator or the stimulation portion of the care engine of FIG. 10, and/or using control signals based on information communicated from sensors, in some examples. In other examples, an external controller may perform the method 900 and communicate with the PG. The control signals may be based on or responsive to sensor signals indicative of the parameters and which cause selective stimulating of the at least one fiber of the glossopharyngeal nerve, as described in connection with at least the method 10 of FIG. 1 and/or examples of FIGS. 2A-15.

In some examples, the control portion 916 (FIG. 9B) comprises a memory 923 which may store machine readable instructions (and/or store information) 924 executable on processor 921. Among other instructions (and/or stored information), in some examples the instructions (and/or stored information) 924 may comprise a body position parameter 929-1, an obstruction parameter 929-2, respiration parameter 929-3, sleep state parameter 929-4, cardiac parameter 929-5, and/or other parameter 929-6. Among other uses, these parameters (alone or in various combinations) may provide or correspond to physiologic signals (and/or information derived therefrom) by which patient care may be provided such as (but not limited to) sensing, delivering therapy, tracking, evaluation, etc. according to various examples of the present disclosure.

As shown at 901 in FIG. 9A, the method 900 comprises selecting SDB care. The SDB care may be set by a care provider and/or may be adjusted over time based on feedback, such as the sensed parameters and/or results of SDB care provided.

In some examples, the selection of SDB care may include selectively stimulating at different target locations of the body to cause implementation of different sensory functions and/or motor functions of the glossopharyngeal nerve, such as stimulating at target locations A, B, and/or C of FIGS. 3A and 3C. In some examples, the selection of SDB care may be set based on where the at least one stimulation element is implanted. For example, the stimulation timing, length, and/or strength of stimulation (e.g., stimulation energy level) may depend on the type of glossopharyngeal-related tissue, such as at least one fiber of the glossopharyngeal nerve that is stimulated and/or on which branch of the glossopharyngeal nerve the stimulation occurs. Further details regarding applying stimulation to at least one target tissue is described later in association with at least FIGS. 10-12H. As specific, and non-limiting examples, to activate the stylopharyngeus muscle via stimulation of at least one efferent fiber of the glossopharyngeal nerve or direct stimulation of the stylopharyngeus muscle, an example stimulation pattern may include a stimulation energy level of 40 Hz and a 50% duty cycle, which may be similar to a stimulation pattern used for activating a genioglossus muscle. An example stimulation pattern to activate the at least one the pharyngeal constrictor muscle via stimulation of at least one efferent fiber of the glossopharyngeal nerve may include a stimulation energy level of between about 2 Hz to about 100 Hz and a 100% duty cycle.

As examples and as previously described, if the provided SDB care includes selective stimulation of at least one afferent fiber of the glossopharyngeal nerve to activate and/or prime a sensory pathway associated with upper airway patency, the stimulation may be provided during a treatment period (e.g., via stimulation pulses according to a duty cycle, steady, closed loop or open loop, etc.) when the patient is in a sleep state. The stimulation may be provided at a stimulation (e.g., energy) level below a threshold to otherwise keep the sensory pathway activated and/or primed without regard to detection of an SDB event(s) and timing of respiration. As previously described, activated, or priming the sensory pathway associated with upper airway patency may invoke a reflex opening activity of the entire array (or substantially the entire array) of upper airway patency-related muscles.

As another example, for SBD care that selectively stimulates at least one efferent fiber of the glossopharyngeal nerve to activate at least one pharyngeal constrictor muscle, the stimulation may be provided during a treatment period as the patient is in a sleep state (e.g., via stimulation pulses according to a duty cycle, steady, closed loop or open loop, etc.). The stimulation may similarly be provided at a stimulation (e.g., energy) level below a threshold to modulate and/or increase pharyngeal muscle tone to reduce collapsibility of the upper airway and without regard to detection of an SDB event and timing of respiration.

As a further example, for the SDB care provided to selectively stimulate at least one efferent fiber of the glossopharyngeal nerve to activate the stylopharyngeus muscle, the stimulation may occur in response to at least one SBD event detection and/or timed with the respiratory cycle of the patient. In some examples, stimulating in response to the SBD event detection is not limited to a single event detection, and may include stimulating in response to detection of an apnea frequency, such as a number of SDB events or apneas per hour (e.g., apnea-hypopnea index (AHI)). For example, the stimulating may occur in response to detecting AHI above a threshold in a treatment period (e.g., one night), such as but not limited to five.

At 910, depending on the selected SBD care (at 901), the method 900 comprises determining if sensed parameters (e.g., respiration parameter 929-3, body position parameter 929-1, etc.) are used to time or otherwise cause the stimulation to provide the selected SDB care. If not, at 920, the method 900 includes identifying the stimulation, such as the stimulation element at or near the target location for the SDB care and/or the amount of stimulation to provide (e.g., steady over a treatment period, duty cycle, threshold energy level, length of time of stimulation). At 930, the SBD care is provided by selectively stimulating the glossopharyngeal-related tissue (e.g., at least one fiber of the glossopharyngeal nerve) according to the selected SDB care.

With this in mind, in some examples, the care may be provided when the patient is in a sleep state, and without determining the user has upper airway obstruction, such as when stimulating at target location A and/or C in FIG. 3A. As an example, the care provided may include electrically stimulating at least efferent fiber of the glossopharyngeal nerve to activate at least one at least one pharyngeal constrictor muscle in response to the patient being in a sleep state, without timing with the respiration cycle and/or without regard to SBD events and without activating coughing and/or trachea closure. In some instances, this stimulation protocol may sometimes be referred to as one example/type of open loop stimulation. In some examples, the at least one efferent fiber of the glossopharyngeal nerve may be stimulated (e.g., steady stimulation or pulsed stimulation according to a duty cycle) at below a threshold level over a treatment period (e.g., during the night and while sleeping). As another example, the care provided may include stimulating at least afferent fiber of the glossopharyngeal nerve to activate and/or prime a sensory pathway associated with upper airway patency, e.g., reflex opening activity, without timing with the respiration cycle and/or without regard to SBD events and without activating coughing and/or trachea closure, as previously described. For example, the at least one afferent fiber of the glossopharyngeal nerve may be steadily stimulated or stimulated at a duty cycle with stimulation at below a threshold level over a treatment period.

If at least one of the sensed parameters is used to time and/or cause the stimulation, at 915, the respective sensed parameters are selected and/or otherwise assessed. And, at 925, based on the selected parameters, the method 900 comprises identifying the timing and/or stimulation to provide, such as identifying respiration information, detection of at least one SDB event (e.g., AHI above a threshold), the target location for the SDB care and/or the amount of stimulation to provide (e.g., supra-threshold level, tone level, etc.). And at 930, the SBD care is provided by selectively stimulating the glossopharyngeal-related tissue (e.g., at least one fiber of the glossopharyngeal nerve) according to the selected SDB care. For example, the stimulation may be timed with (e.g., relative to) a fiducial of a respiration waveform (e.g., cycle) of the patient. In some examples, timing the stimulation with a fiducial(s) of the respiration waveform may include triggering and/or synchronizing the stimulation with the fiducial(s) of the respiration waveform.

As an example, care may be provided in response to identifying the user is in a sleep state (e.g., parameter 929-4) and/or has a rate of upper airway obstruction above a threshold (e.g., AHI above a threshold, and parameter 929-2), such as when stimulating at target location B in FIG. 3A. In some examples, the care provided may include electrically stimulating at least efferent fiber of the glossopharyngeal nerve to activate at least one stylopharyngeus muscle and as timed with the inspiration phase of the respiration cycle and without activating coughing and/or trachea closure, as previously described.

As described above and in accordance with some examples, the method 900 may comprise sensing various parameters. As shown by FIG. 9B, the different parameters may include body position parameter 929-1, obstruction parameter 929-2, respiration parameter 929-3, sleep state parameter 929-4, cardiac parameter 929-5, and/or others 929-6. Different sensors may sense and communicate the sensor signals to the control portion 916 to determine the parameters from the sensor signals. In some examples, sensed physiologic information in addition to, or instead of, the respiratory waveform, body position, and/or sleep state (or other sleep information may be used to select SDB care for the patient. At least some other/additional physiologic information, as well as the respiration information, body position, and/or sleep information, is further described in association with at least FIG. 9C and FIG. 11.

In some examples, the obstruction parameter 929-2 may include or be used to determine collapse patterns associated with SDB. Example collapse patterns include an AP collapse pattern, a concentric collapse pattern, a lateral collapse pattern, and an AP—lateral collapse pattern. In some examples, the different collapse patterns may be alleviated by stimulation of certain nerves or stimulation of combination of certain nerves. In accordance with various examples, the selective stimulation of an afferent fiber of glossopharyngeal nerve is used to activate and/or prime a sensory pathway associated with reflex opening of the upper airway. Once the sensory pathway is active or primed, a comprehensive reflex/opening effect occurs due to increased sensitivity of the pathway which may alleviate a wider range of type/degrees of collapse patterns as compared to stimulating a single class/type of efferent fiber innervating upper airway patency-related muscles such as, but not limited to, hypoglossal nerve or infrahyoid muscle-innervating nerve, as some examples. For example, by stimulating the single glossopharyngeal nerve (or portion thereof), via its sensory pathway, the therapy may invoke a comprehensive response of most or all of the upper airway patency-related muscles as part of the reflex opening activity.

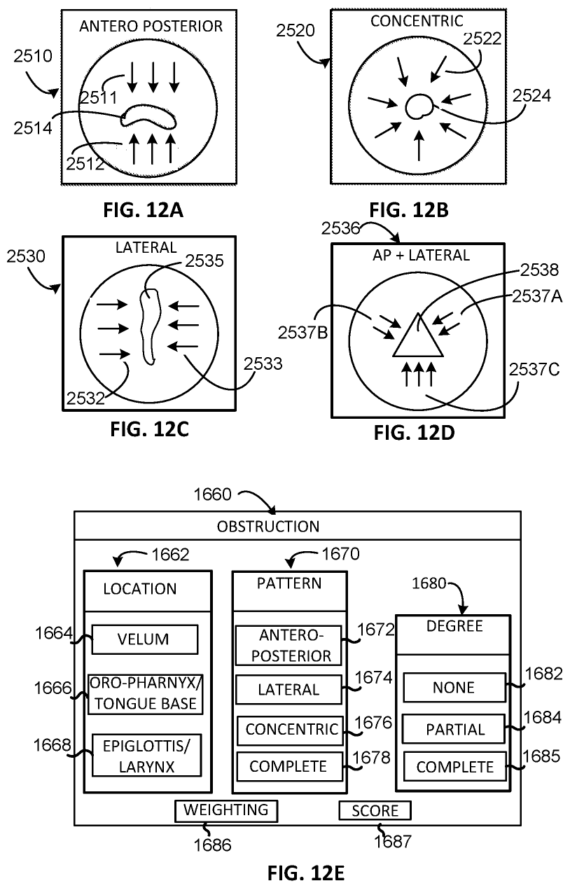

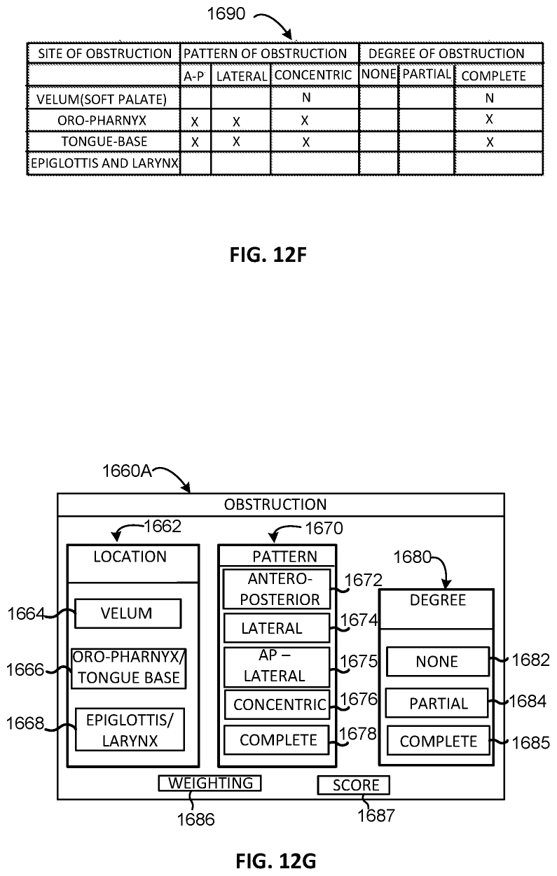

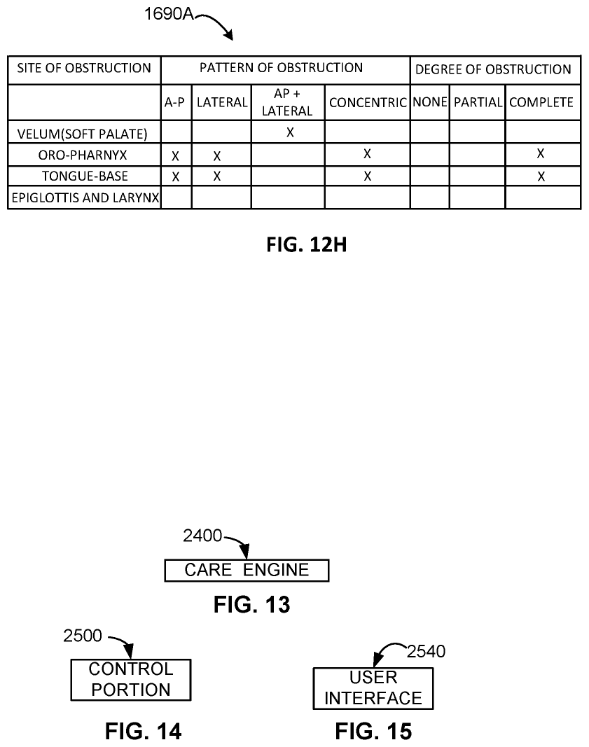

In some such examples, the SDB care to provide to a patient may be selected based on the exhibited collapse pattern of the upper airway. For example, based on the collapse pattern exhibited, at least one upper airway patency-related tissue may be stimulated. In some examples, a target location and/or target fiber(s) of the glossopharyngeal nerve (or other nerves) may be selected for application of stimulation based on the collapse pattern and/or level of collapse, such as further illustrated in connection with FIGS. 12A-12D. Patients exhibiting particular collapse patterns may be more responsive to different stimulation. The responsiveness may be patient specific or general across many patients. Accordingly, the presence of or lack of a particular collapse pattern of obstruction and, optionally, a level or degree of collapse or obstruction of the upper airway, may be used to select the SDB care to provide to the user.

It will be understood that various patterns of collapse occur at different levels of the upper airway portion and that the level of the upper airway in which a particular pattern of collapse appears may vary from patient-to-patient.

In addition to observing such collapse patterns and/or other collapse patterns, at least some aspects of such collapse patterns may be measured, such as via impedance sensing using implanted electrodes (e.g., sensing elements and/or stimulation elements), using externally applied arrays of electrodes, etc.

As previously noted in relation to the method 900 of FIG. 9A, FIG. 9B illustrates an example control portion, which may be used to implement the method 900 of FIG. 9A and/or any of the methods described herein. In some examples, the control portion 916 of FIG. 9B may be implemented by pulse generator described and/or illustrated by any of FIGS. 1-8 and/or include an implementation of, and/or at least some of substantially the same features and attributes as, control portion 1300 of FIGS. 6A-8 and/or the stimulation portion as further described by FIG. 11.

As shown by FIG. 9B, the control portion 916 includes a processor 921 and memory 923 that stores machine readable instructions. The processor 921 may execute the machine readable instructions stored on the memory 923, which when executed cause the processor 921 to perform the above-identified actions, including but not limited to the method 10 of FIG. 1, the method 900 of FIG. 9A, and/or any of the variation described in connection with FIGS. 2A-8.

The control portion 916 may be in communication with the sensors 927-1, 927-2, 927-3 via a wired or wireless communication link, among other components. In response to received sensor signals, the processor 921 may identify various parameters 929-1, 929-2, 929-3, 929-4, 929-5, 929-6 of the patient, and may store the parameters on memory 923. In some examples, the processor 921 may use the stored parameters to determine SDB care to provide to the patient at a particular date and time.

The sensors 927-1, 927-2, 927-3 may include a variety of different types of sensors. Example sensors include an acceleration sensor, a pressure sensor, an impedance sensor, an airflow sensor, a radio frequency sensor, electromyography (EMG) sensor, electrocardiogramaensor, ultrasonic, acoustic sensor, image sensor, and/or other types of sensors. Each of the sensors may be implemented as an external sensor and/or an implantable sensor.

It will be understood that any of the parameters (929-1, 929-2, 929-3, 929-4, 929-5, 929-6) may be determined, tracked, etc. via one or more of the different sensor modalities (alone or in combination) as described in association with sensors 927-1, 927-2, 927-3, with some more specific examples further described below.

An acceleration sensor may include accelerometer (e.g., a multi-axis accelerometer such as a three-axis or six-axis accelerometer), a gyroscope, etc., and may be used to identify the body position (parameter 929-1), respiration information (parameter 929-3, e.g., waveform morphology, respiratory cycle, phase information, etc.), and/or sleep state (parameter 929-4) of the patient.

The acceleration sensor may be used to sense an amount of acceleration and, therefrom, to identify body motion and posture, e.g., body position parameter 929-1. Example body motions include movement in a vector or a direction (e.g., walking, running, biking), rotational motions (e.g., twisting), sliding motions, and changes in posture (e.g., change from upright position to a sitting or supine position), among other movements. The motion may be sensed relative to a gravity vector, such as an earth gravity vector and/or a vertical baseline gravity vector for calibrating the data. In various examples, the sensed forces may be processed to determine a posture of the patient. As used herein, posture includes and/or refers to a position or bearing of the body. The term “posture” may sometimes be referred to as “body position”. Example postures include upright or standing position, supine position (e.g., generally horizontal body position), a generally supine reclined position, sitting position, etc.

In some examples, the acceleration sensor may be used to sense additional physiological data. The additional physiological data may include additional physiological parameters, such as (but not limited to) cardiac signals/information per parameter 929-5 and/or respiration signals/information per parameter 929-3. As further described herein, the respiration information may be determined based on rotational movements of a portion of a chest wall of the patient during breathing. For example, the acceleration sensor may be used to determine respiration information, cardiac information, detection of SDB events, sleep information, and/or other information or be implemented according to at least some of substantially the same features and attributes as described within: U.S. Pat. No. 11,324,950, granted on May 5, 2033, entitled “ACCELEROMETER-BASED SENSING FOR SLEEP DISORDED BREATHING (SDB) CARE”; U.S. Patent Publication No. US2023/0119173, published on Apr. 20, 2023, and entitled “RESPIRATION DETECTION”; U.S. Patent Publication No. US2023/0277121, published on Sep. 7, 2023, and entitled “DISEASE BURDEN INDICATION”; and PCT Publication No. WO2022/261311, published on Dec. 15, 2022, and entitled “RESPIRATION SENSING”, the entire teachings of which are each incorporated herein by reference in their entirety.

In some examples, a pressure sensor may sense pressure, sound, and/or pressure waves. The pressure, sound, and/or pressure waves may be indicative of and/or used to determine different parameters, such as respiration information (parameter 929-3, e.g., respiration cycle, inspiration, exhalation, respiration rate), cardiac information (e.g., QRS complex, heart rate, heart rate variability), SDB obstruction (parameter 929-2), among other parameters. For example, a pressure sensor may comprise an implantable respiratory sensor. In some examples, pressure sensor comprises piezoelectric element(s), although examples are not so limited.

In some examples, an airflow sensor may be used to sense respiration information/parameter 929-3, SBD obstruction/parameter 929-2 or other SBD related information, sleep quality information, etc. In some instances, airflow sensor detects a rate or volume of upper respiratory airflow.

In some examples, an impedance sensor may sense a bio-impedance signal. The bio-impedance signals may be indicative of and/or used to determine parameters, such as an SDB obstruction or events (parameter 929-2), cardiac information (parameter 929-5, e.g., ECG signal, derived cardiac metrics), respiration information (parameter 929-3), including or indicative of the of the inspiratory and/or expiratory phases, and inspiratory rate, among other parameters. The impedance sensor may be implemented as various sensors distributed about the upper body, whether the sensors are internal and/or external to the patient.

In some examples, a radio frequency (RF) sensor is used to enable non-contact sensing of various additional physiologic parameters and information, such as but not limited to respiration information (parameter 929-3), cardiac information (parameter 929-5), motion/activity, and/or sleep information (parameter 925-6, e.g., sleep quality, or other). In some examples, RF sensor determines chest motion based on Doppler principles, which may be used to sense respiration information (parameter 929-3), cardiac information (parameter 929-5), etc. The RF sensor may be embodied as the electromagnetic field sensor, in some examples.

In some examples, the at least one sensor includes an optical sensor. The optical sensor may sense heart rate and/or oxygen saturation via pulse oximetry, and/or oxygen desaturation index (ODI).

An EMG sensor may be used to record and evaluate electrical activity produced by muscles, whether the muscles are activated electrically or neurologically. In some instances, the EMG sensor is used to sense respiration information (parameter 929-3), such as but not limited to, respiration rate, phase information, etc., and/or SDB obstruction (parameter 929-2).

In some examples, an ECG sensor may be used which produces an ECG signal. In some instances, the ECG sensor comprises a plurality of electrodes distributable about a chest region of the patient and from which the ECG signal is obtainable. In some examples, ECG sensor is used to detect SDB obstruction (parameter 929-2), respiration information (parameter 929-3), and/or cardiac information (parameter 929-5).

In some examples, an ultrasonic sensor may be used to detect an ultrasonic signal. In some instances, ultrasonic sensor is locatable in close proximity to an opening (e.g., nose, mouth) of the patient's upper airway and via ultrasonic signal detection and processing, may sense exhaled air to enable determining at least respiration information, such as respiration information (parameter 929-3), sleep quality information, SDB information, such as obstruction parameter 929-2, etc.

In some examples, acoustic sensor comprises piezoelectric element(s), accelerometers, etc., which sense acoustic vibration. In some implementations, such acoustic vibratory sensing may be used to detect snoring which may be indicative of at least SDB obstruction (parameter 929-2), respiration information (parameter 929-3, including SDB information in addition to obstruction).

FIG. 9C is a block diagram schematically representing an example sensing portion of an example device and which may be used to implement the method 900 of FIG. 9A. In some examples, an example method may employ an example SDB care device (e.g., including stimulation element 201 in FIGS. 2B-2C) comprising the sensing portion 2000 to sense physiologic information and/or other information, with such sensed information relating to care of a wide variety of physical conditions such as, but not limited to, SDB care, pelvic care, cardiac care, among other uses. In some examples, the sensing portion 2000 of FIG. 9C may be implemented by PG described and/or illustrated by any of FIGS. 1-8 and/or may include an implementation of, and/or at least some of substantially the same features and attributes as, control portion 1300 of FIGS. 6A-8. In some examples, the sensing portion 2000 may be implemented by a component/device external to the PG and/or SDB care device, such as illustrated in connection with FIG. 11.

The sensed information may be used to implement at least some of the example methods and/or examples devices described in association with at least FIGS. 1-9B. It will be understood that the sensing portion 2000 may be implemented as single sensor or multiple sensors, and may comprise a single type of sensor or multiple types of sensing. In addition, it will be further understood that the various types of sensing schematically represented in FIG. 9C may correspond to a sensor and/or a sensing modality.

In some examples, the sensed information may refer to physiologic signals (e.g., biosignals) and/or metrics which may derived from such physiologic signals. For example, among other sensed physiologic signals, one physiologic signal may comprise respiration (parameter 2005 in FIG. 9C), from which various metrics may be derived such as, but not limited to, respiratory rate, respiratory rate variability, respiratory phase, rate times volume, waveform morphology, and more. The respiration information may be sensed via at least one of the sensing modalities described below (and/or other sensing modalities) such as, but not limited to, accelerometer 2026, ECG 2020, impedance 2036, pressure 2037, temperature 2038, acoustic 2039, and/or other sensing modalities, at least some of which are further described below. The respiration information may be used for a wide variety of purposes such as, but not limited to, timing stimulation relative to respiration, disease burden, sleep-wake status, arousals, etc. In some such examples, the detection of disease burden may comprise detection of SDB events, which may be used in determining, assessing, etc. therapy outcomes such as, but not limited to, AHI.

In some examples, the sensed physiologic information may comprise cardiac information (2006) obtained from a cardiac signal and from which various metrics may be derived such as, but not limited to, heart rate (HR), heart rate variability (HRV), P-R intervals, waveform morphology, and more. One example of a cardiac signal may comprise an ECG signal, as represented at 2020 in FIG. 9C. Accordingly, the cardiac information and/or signal may be sensed via at least one sensing modality further described below (and/or other sensing modalities) such as, but not limited to, cardiac sensor 2023, accelerometer 2026, ECG 2020, EMG 2022, impedance 2036, pressure 2037, temperature 2038, and/or acoustic 2039. In some examples, the sensed physiologic information (e.g., via sensing portion 2000) may comprise a wide variety of physiologic information other (2007) than respiration and/or cardiac information, with at least some examples.

The sensed physiologic signals and/or information (e.g., respiration 2005, cardiac 2006, and/or other information 2007) may be used for a wide variety of purposes such as, but not limited to, determining sleep-wake status (e.g., various sleep onset determinations), timing stimulation relative to respiration, determining disease burden, determining arousals, etc. In some such examples, the determination of disease burden may comprise detection of SDB events, which may be used in determining, assessing, etc. therapy outcomes such as, but not limited to, AHI, as well as titrating stimulation parameters, adjusting sensitivity of sensing the physiologic information, etc.

For instance, in one non-limiting example, an ECG sensor 2020 in FIG. 9C may comprise a sensing element (e.g., electrode) or multiple sensing elements arranged relative to a patient's body (e.g., implanted in the transthoracic region) to obtain ECG information. In some examples, the ECG information may comprise one example implementation to obtain cardiac information, including but not limited to, HR 2025A, HRV 2025B, and other cardiac parameters 2025C, which may be used (with or without other information) in determining delivering stimulation therapy and associated sensing (e.g., inputs) for determining effectiveness of the therapy and/or implementing the therapy, as described throughout the examples of the present disclosure.

However, in some instances, the ECG sensor 2020 may represent ECG sensing element(s) in general terms without regard to a particular manner in which sensing ECG information may be implemented.

In some examples in which multiple electrodes are employed to obtain an ECG signal, an ECG electrode may be mounted on or form at least part of a case (e.g., outer housing) of a stimulation support portion (which may comprise an IPG in some examples). In such instances, other ECG electrodes are spaced apart from the ECG electrode associated with the stimulation support portion. In some examples, at least some ECG sensing electrodes also may be employed to deliver stimulation to a nerve or muscle, such as but not limited to, an upper airway patency-related nerve (e.g., hypoglossal nerve) or other nerves or muscles (e.g., glossopharyngeal-related tissue).

In some examples, other types of sensing may be employed to obtain cardiac information (including but not limited to heart rate and/or heart rate variability), such as a cardiac sensor 2023 shown in FIG. 9C, which may comprise at least one of a ballistocardiogram sensor(s), seismocardiogram sensor(s), and/or accelerocardiogram sensor(s). In some examples, such sensing is based on and/or implemented via accelerometer-based sensing such as further described below in association with accelerometer 2026.

In some examples in which the cardiac sensor 2023 comprises a ballistocardiogram sensor, the sensor 2023 may sense cardiac information caused by cardiac output, such as the forceful ejection of blood from the heart into the great arteries that occurs with each heartbeat. The sensed ballistocardiogram information may comprise HR 2025A, HRV 2025B, and/or additional cardiac morphology 2025C. In some examples such ballistocardiogram-type information may be sensed from within a blood vessel in which the sensor (e.g., accelerometer) senses the movement of the vessel wall caused by pulsations of blood moving through the vessel with each heartbeat. This phenomenon may sometimes be referred to as arterial motion.

In some examples in which the cardiac sensor 2023 comprises a seismocardiogram sensor, the sensor 2023 may provide cardiac information which is similar to that described for ballistocardiogram sensor, except for being obtained via sensing vibrations, per an accelerometer (e.g., single or multi-axis), in or along the chest wall caused by cardiac output. In particular, the seismocardiogram measures the compression waves generated by the heart (e.g., per heart wall motion and/or blood flow) during its movement and transmitted to the chest wall. Accordingly, the sensor 2023 may be placed in the chest wall.

In some such examples of sensing per sensor 2023, such methods and/or devices also may comprise sensing a respiratory rate and/or other respiration information.

In some examples the sensing portion 2000 may comprise an electroencephalography (EEG) sensor 2012 to obtain and track EEG information. In some examples, the EEG sensor 2012 may also sense and/or track central nervous system (CNS) information in addition to sensing EEG information. In some examples, the EEG sensor(s) 2012 may be implanted subdermally under the scalp or may be implanted in a head-and-neck region otherwise suitable to sense EEG information. Accordingly, the EEG sensor(s) 210 are located near the brain and may detect frequencies associated with electrical brain activity.

In some examples, a sensing element used to sense EEG information is chronically implantable, such as in a subdermal location (e.g., subcutaneous location external to the cranium skull), rather than an intracranial position (e.g., interior to the cranium skull). In some examples, the EEG sensing element is placed and/or designed to sense EEG information without stimulating a vagus nerve at least because stimulating the vagal nerve may exacerbate sleep apnea, particularly with regard to obstructive sleep apnea. Similarly, the EEG sensing element may be used in a device in which a stimulation element delivers stimulation to a hypoglossal nerve or other upper airway patency-related nerve without stimulating the vagus nerve in order to avoid exacerbating the obstructive sleep apnea.

In some examples, sensed EEG information may be used as part of (or solely in) making a sleep-wake determination, such as sleep onset, and wake onset. Among other uses, this sleep-wake information may help provide overall sleep hours, which may comprise part of therapy outcome, in some examples.

In some examples, sensed EEG information may be used to detect sleep stages during sleep. Among other uses, this sensed sleep stage may help determine an absolute amount or relative amount of deep sleep, REM sleep per night, and/or other sleep metrics. For instance, such information may be used to evaluate whether a particular stimulation setting corresponds to a patient's most therapeutic stimulation energy settings/parameters based on (at least or in part) the recognition more deep sleep typically corresponds to the most or more therapeutic stimulation energy settings whereas less deep sleep typically corresponds to lesser therapeutic stimulation energy settings.

In some examples, sensed EEG information may be used to detect arousals, which may comprise one aspect of determining therapy outcome. Among other uses, the detection of more arousals may provide an indication of the patient exhibiting more daytime sleepiness, which in turn may lead to adjustments to stimulation solution settings (e.g., values of stimulation energy parameters) in order to minimize arousals.

In some examples, the above-described aspects regarding the use of sensed EEG information may be combined in whole, or part, to provide an overall sleep efficiency parameter. In some such examples, the sleep efficiency parameter may be based on: 1) sleep duration; 2) sleep depth; and/or 3) events (e.g., number of arousals). In some examples, the sleep efficiency parameter may be compared to a reference sleep efficiency parameter such as (but not limited to): 1) a reference sleep duration (e.g., 8-9 hours); 2) a reference sleep depth (e.g., a minimum duration of deep sleep and REM sleep; and/or 3) few or no arousals.

In some examples the sensing portion 2000 may comprise an electromyogram (EMG) sensor 2022 to obtain and track EMG information. In some examples, the sensed EMG signals may be used to identify sleep, respiration information (e.g., respiratory phase information) and/or obstructive events. In some examples, the detected EMG information may be used to detect arousals and/or overall patient movement. These examples of determining and/or using sensed EMG information may be used as part of determining patient metrics (e.g., therapy outcome, usage, other) by which stimulation energy parameters may be determined, adjusted, etc. in order to maintain and/or improve those patient metrics according to various examples of the present disclosure.

In some examples, any one or a combination of the various sensing modalities (e.g., EEG, EMG, etc.) described in association with FIG. 9C may be implemented via a single sensing element 2014.

In some examples, the sensing portion 2000 may comprise an accelerometer 2026. In some examples, the accelerometer may comprise a single axis accelerometer while in some examples, the accelerometer may comprise a multiple axis accelerometer.

Among other types and/or ways of sensing information, the accelerometer sensor(s) 2026 may be employed to sense or obtain a ballistocardiogram, a seismocardiogram, and/or an accelerocardiogram (see cardiac sensor 2023 and related disclosure), which may be used to sense (at least) HR 2025A and/or HRV 2025B (among other information such as respiratory rate in in some instances), which may in turn may be used as part of determining respiration information, cardiac information, as described throughout the examples of the present disclosure. In some examples, this sensed information also may be used in determining sleep-wake status.

In some examples, the accelerometer 2026 may be used to sense activity, posture, and/or body position as part of determining a patient metric, the sensed activity, posture, and/or body position may sometimes be at least partially indicative of a sleep-wake status, which may be used as part of automatically initiating, pausing, and/or terminating stimulation therapy.

In some examples, the sensing portion 2000 may comprise an impedance sensor 2036, which may sense transthoracic impedance or other bioimpedance of the patient. In some examples, the impedance sensor 2036 may comprise a plurality of sensing elements (e.g., electrodes) spaced apart from each other across a portion of the patient's body. In some such examples, one of the sensing elements may be mounted on or form part of an outer surface a housing of a stimulation support portion (e.g., 133 in FIG. 2D) or other implantable sensing monitor, while other sensing elements may be located at a spaced distance from the stimulation support portion and/or stimulation electrode arrangement. In at least some such examples, the impedance sensing arrangement integrates all the motion/change of the body (e.g., such as respiratory effort, cardiac motion, etc.) between the sense electrodes (including the case of the IPG when present). Some examples implementations of the impedance measurement circuit include separate drive and measure electrodes to control for electrode to tissue access impedance at the driving nodes. Such impedance sensing may be used for other purposes.

In some examples, the sensing portion 2000 may comprise a pressure sensor 2037, which senses respiration information, such as but not limited to respiratory cyclical information. In some examples, the pressure sensor 2037 may be located in direct or indirect continuity with respiratory organs or airway or tissues supporting the respiratory organs or airway in order to sense respiration information.

In some examples, one sensing modality within sensing portion 2000 may be at least partially implemented via another sensing modality within sensing portion 2000.

In some examples, sensing portion 2000 may comprise an acoustic sensor 2039 to sense acoustic information, such as but not limited to cardiac information (including heart sounds), respiration information, snoring, etc.

In some examples, sensing portion 2000 may comprise body motion parameter 2035 by which patient body motion may be detected, tracked, etc. The body motion may be detected, tracked, etc. via a single type of sensor or via multiple types of sensing. For instance, in some examples, body motion may be sensed via accelerometer 2026 and in some examples, body motion may be sensed via EMG 2022 and/or other sensing modalities.

In some examples, the sensing portion 2000 in FIG. 9C may comprise a body position/posture parameter 2042 and/or body motion parameter 2035 to sense and/or track sensed information regarding posture, which also may comprise sensing of body position, activity, etc. of the patient. This sensed information may be indicative of an awake or sleep state of the patient in some examples. In some such examples, such information may be sensed via accelerometer 2026 as mentioned above, and/or other sensing modalities. In some examples, such posture information (and/or body position, activity) may be used sometimes alone and/or in combination with other sensing information to determine a patient metric. As described elsewhere herein, in some examples posture may be considered as one of several parameters when determining a probability of sleep (or awake). In some such examples, the sleep-wake status may be used to initiate, pause, and/or terminate stimulation therapy within a nightly treatment period.

In addition or alternatively, sensing activity, motion, and/or body position (e.g., posture) may be used to track a relative degree to which a patient is more active or less active during daytime hours, which may comprise one objective measure of therapy outcome because if the patient is sleeping better at night due to a desirable stimulation solution settings (e.g., values of stimulation energy parameters) which better control SDB, the patient may be much more active during daytime (non-sleep) hours as compared to a baseline in which their sleep disordered breathing was poorly controlled (corresponding to inferior stimulation energy settings) or not controlled at all. Similarly, sensing activity and/or motion as described herein also may be used to detect if the patient tends to falls asleep during daytime (e.g., non-sleep) hours, which may be an objective therapy outcome parameter by which stimulation energy parameters (and associated usage, and other therapy outcome parameters) may be evaluated and potentially adjusted according to at least some examples of the present disclosure. This objective therapy outcome information also may be used in conjunction with subjective therapy outcome information such as, but not limited to, the Epworth Sleepiness Scale (ESS) and/or other forms of patient input regarding the patient's perceived daytime sleepiness, daytime functional ability, perceived sleep quality, etc.

In some examples, the sensing portion 2000 may comprise an other parameter 2041 to direct sensing of, and/or receive, track, evaluate, etc. sensed information other than the previously described information sensed via the sensing portion 2000.

As further shown in FIG. 9C, in some examples the sensing portion 2000 may comprise a temperature sensor 2038. In some example methods, sensing a change in temperature (such as via sensor 2038) during a treatment period may be used to identify SDB behavior. In some such examples, additional sensed information (as described in examples of the present disclosure) may be used in addition to sensed temperature to identify sleep SDB behavior. In some examples, smaller yet detectable temperature changes within a treatment period may be used to at least partially determine a patient metric. For instance, a detectable temperature change may be sensed as a result of patient exertion to breathe in response to an apnea event, given the greater muscular effort in attempting to breathe.

In some examples, at least some of the sensors and/or sensor modalities described in association with FIG. 9C may be incorporated within or on a stimulation element (e.g., 201 in FIGS. 2B-2C) which comprise at least some implantable components, in some examples.

FIG. 10 is a block diagram schematically representing a stimulation portion of a care engine. In some examples, the stimulation portion 1600 may comprise an example implementation of, and/or at least some of substantially the same features and attributes as, the PG, IPG, and/or the control portion (e.g., FIGS. 1-9C) of the present disclosure. Accordingly, the various functions and parameters of the stimulation portion 1600 of FIG. 10 may be implemented in a manner supportive of, and/or complementary with, the various functions, parameters, portions, etc., of any of the devices and control portions and/or various functions, parameters, portions, etc., relating to stimulation throughout examples of the present disclosure. In some examples, the stimulation portion 1600 may include an implementation of at least a portion of the care engine 1311 of FIG. 6A and/or care engine/control portion 916 in FIG. 9B.

In some examples, different target tissue may be stimulated using at least one stimulation element, as described herein. The different target tissue may be stimulated at the same time (e.g., concurrently) or at different times and in response to different parameters, such as those described and illustrated in connection with at least FIGS. 9A-9C.

In some examples, any of the methods, apparatuses and/or devices may be used to target different target tissue, including those described by FIGS. 1-15. For example, the method 10 of FIG. 1 may further comprise performing at least one of: (i) stimulating at least one additional upper airway patency-related tissue, (ii) stimulating at least one diaphragm-related tissue, and (iii) activating an external breathing therapy device. Example upper airway patency-related tissue includes the hypoglossal nerve and/or muscles innervated by the hypoglossal nerve, an additional portion of the glossopharyngeal nerve or other glossopharyngeal-related tissue, infrahyoid muscle-related nerve, and infrahyoid strap muscle, among other upper airway patency-related tissue. The combination of stimulating upper airway patency-related tissue, pulling the pulling the thyroid cartilage and/or hyoid bone, and/or activating the external breathing therapy device may be used to treat OSA as well as multiple type SDB when such upper airway patency-related tissue therapies are combined with the stimulation of diaphragm-related tissue (e.g., phrenic nerve and/or diaphragm), which is used to treat central sleep apnea (CSA). In some examples, other nerve and/or tissue may be stimulated or treated.

As shown by FIG. 10, in some examples, via target tissue parameter 1610, stimulation may be delivered to selectable target tissue such as, but not limited to, upper airway patency-related tissue. In some examples, the upper airway patency-related tissue may comprise at least one fiber of the glossopharyngeal nerves and/or muscle (e.g., stylopharyngeus muscle) innervated by the glossopharyngeal nerve, as described above. In some examples, the upper airway patency-related tissue may comprise a hypoglossal nerve and/or muscle (e.g., genioglossus muscle) innervated by the hypoglossal nerve to cause contraction of at least the protrusion muscles and to thereby cause protrusion of the tongue to increase and/or maintain upper airway patency. In some examples, the upper airway patency-related tissue may comprise infrahyoid muscle-related nerves which includes a nerve(s) innervating at least one infrahyoid strap muscle (e.g., thyrohyoid, omohyoid, sternohyoid, and/or sternothyroid), and may include an ansa cervicalis-related nerve. The infrahyoid muscle-related nerves may sometimes be referred to as infrahyoid muscle-innervating nerves. In some examples, target tissue may include any other muscles which affect and/or promote upper airway patency, and/or nerves which innervate such muscles. In some examples, target tissue includes a combination of nerves and/or muscles such as, but not limited to, terminal fiber ends of nerves where a nerve ending terminates into (or at) the muscle being innervated. In some examples, such as when it is desired to treat CSA (not OSA), target tissue may include diaphragm-related tissue, such as the phrenic nerve and/or diaphragm, among other tissue.

In some examples, in addition to or instead of selecting different tissue (e.g., nerves and/or muscles) for stimulation, the target tissue parameter 1610 may comprise adjusting stimulation parameters via selecting between (or using a combination of) various locations along a nerve or muscle, such as stimulating multiple different sites along a particular nerve (e.g., glossopharyngeal nerve), with some stimulation sites being more distal and some being more proximal.

In some examples, in addition to or instead of selecting different tissues for stimulation and/or for mechanical maneuvering, the target tissue parameter 1610 may comprise adjusting stimulation parameters via selecting between (or using a combination of) different fascicles within a particular nerve in order to selectively stimulate target efferent and/or afferent fibers while omitting (or minimally impacting) stimulation of other, non-target fibers.

In some examples, the stimulation portion 1600 may implement stimulation according to a bilateral parameter 1612 in which stimulation is applied to a target tissue on both sides (e.g., left and right) of the patient's body. In some such examples, the bilateral stimulation may be delivered to the same nerve or other tissue (e.g., glossopharyngeal nerve) on both sides of the body. However, in some examples, the bilateral stimulation may be delivered to different nerves (e.g., glossopharyngeal nerve, hypoglossal nerve, infrahyoid muscle-innervating nerve) or different muscles, such as stimulating one nerve (e.g., hypoglossal nerve) on a left side of the body while stimulating another nerve (e.g., glossopharyngeal nerve) on a right side of the body, or vice versa. In some examples, in which CSA may be treated, such as part of treating multi-type sleep apnea (e.g., both OSA and CSA), stimulation of a phrenic nerve (or diaphragm muscle) may be included in a bilateral stimulation method to implement the stimulation aspects directed to treating CSA.

In some examples, the bilateral parameter 1612 may be implemented in a manner complementary with the alternating parameter 1632, simultaneous parameter 1634, or demand parameter 1636 of multiple function 1630, as further described below.

In some examples, the stimulation portion 1600 may comprise a multiple function 1630 by which various stimulation parameters may be implemented in dynamic arrangements. In some such examples, the stimulation portion 1600 may comprise an alternating parameter 1632 by which stimulation of one target tissue (e.g., glossopharyngeal nerve) may be alternated with stimulation of at least one other target tissue (e.g., hypoglossal nerve). However, the alternating parameter 1632 also may be applied in combination with the bilateral parameter 1612 to apply stimulation to the same tissue (or different tissue) on opposite sides of the body in which stimulation may be applied on a left side of the body and then applied on the right side of the body in an alternating manner.

In some examples, the stimulation portion 1600 may comprise a simultaneous parameter 1634 by which stimulation may be applied simultaneously to at least two different target tissues. In some examples, the at least two different target tissues comprise two different nerves, such as the hypoglossal nerve and the glossopharyngeal nerve. However, in some examples, the at least two different target tissues may comprise two different locations along the same nerve, two different fascicles of the same nerve, and/or muscles. In some examples, the simultaneous parameter 1634 may apply stimulation per bilateral parameter 1612 simultaneously on opposite sides of the body to the same nerve (e.g., glossopharyngeal nerve) or different nerves.

In some examples, the stimulation portion 1600 may comprise a demand parameter 1636 by which stimulation may be applied to at least one target tissue on a demand basis. For example, stimulation may be applied to one nerve (e.g., hypoglossal nerve) which may be sufficient to achieve the patient metric (e.g., therapy outcome and/or usage) for most nights, for most sleeping positions (e.g., left and right lateral decubitis, prone), etc., but may become insufficient for some nights (e.g., after consuming alcohol or certain drugs which relax upper airway muscles), some sleeping positions (e.g., supine). In the latter situation, in order to achieve the target patient metric, via the demand parameter 1636, stimulation of a different tissue (e.g., glossopharyngeal nerve) may be implemented in addition to, or instead of, stimulation of the first tissue (e.g., hypoglossal nerve) which was previously being stimulated. In some examples, the first or primary nerve being stimulated may be a nerve other than the hypoglossal nerve such as, but not limited to, the glossopharyngeal nerve or an infrahyoid muscle-innervating nerve.

In some examples, the stimulation portion 1600 also may further implement at least some aspects of the control portion of FIGS. 6A-9B and/or according to at least one of a closed loop parameter 1620, open loop parameter 1622, and nightly titration parameter 1624.

In some examples, the stimulation portion 1600 comprises a closed loop parameter 1620 to deliver stimulation therapy based on sensed patient physiologic information and/or other information (e.g., environmental, temporal, etc.). In some such examples, via the closed loop parameter 1620, the sensed information may be used to control the particular timing of the stimulation according to respiration information, in which the stimulation pulses are triggered by or synchronized with specific portions (e.g., inspiratory phase) of the respiratory cycle(s). In some such examples and as previously described, the respiration information and/or other information used with the closed loop parameter 1620 may be determined via the sensors, sensing elements, devices, sensing portions, as previously described in association with at least FIGS. 9B-9C.

In some examples, with or without timing stimulation relative to sensed respiration information, the closed loop mode (1620) may comprise delivering stimulation therapy in response to sensed disease burden, such as the average number of apnea events per a time period (e.g., AHI of average number of apnea events per hour) and/or other therapy outcome metrics (e.g., arousals, patient feedback, ESS and/or other metrics). For example, for some periods of time within a nightly treatment period or over the course of several days/weeks, a patient may experience few SDB events (e.g., apnea events), such that therapy may not be delivered. However, upon the patient beginning to experience SDB at a level high enough to warrant therapy, then via the closed loop parameter 1620, stimulation therapy may be delivered to achieve a therapy outcome and/or usage meeting a criteria per the examples of at least FIGS. 1-11.

In some examples, the stimulation portion 1600 comprises an open loop parameter (e.g., 1622 in FIG. 10) by which SDB therapy (e.g., use) is applied without a feedback loop of sensed physiologic information. In some such examples, in an open loop mode the stimulation therapy is applied during a treatment period without (e.g., independent of) information sensed regarding the patient's sleep quality, sleep state, respiratory phase, AHI, etc. In some such examples, in an open loop mode the stimulation therapy is applied during a treatment period without (e.g., independent of) particular knowledge of respiration information.

In some examples, the stimulation portion 1600 comprises a nightly titration parameter 1624 by which an intensity of the SDB therapy may be titrated (e.g., adjusted) to be more intense (e.g., higher amplitude, greater frequency, and/or greater pulse width) or to be less intense within a nightly treatment period. However, it will be understood that the previously described examples in association with at least FIGS. 1-9C may be performed without (e.g., independent of) a nightly titration parameter 1624 and instead be based on titration according to a time period parameter of more than a day, such as supra-day time period. Accordingly, in some examples, guiding therapy per a patient metric in examples of the present disclosure may be implemented solely according to time period of more than a nightly treatment period.

In some such examples, the nightly titration parameter 1624 may be implemented according to at least some aspects of the example methods and/or example devices of FIGS. 1-9C, for example, depending on the sleep state of the patient. Accordingly, in some examples, the titration parameter may be implemented as automatic titration while in some examples, the titration parameter may be implemented via manual titration by a patient (or clinician). In some examples, the titration parameter may be implemented via combination of patient/manual titration and automatic titration to guide the patient in a manner complementary with their manual titration.

In some such examples, such titration may be implemented at least partially based on sleep quality, which may be obtained via sensed physiologic information, in some examples. It will be understood that such examples may be employed with synchronizing stimulation to sensed respiration information (e.g., closed loop stimulation) or may be employed without synchronizing stimulation to sensed respiration information (e.g., open loop stimulation).

In some examples, at least some aspects of the titration parameter 1624 of the stimulation portion 1600 and/or at least some aspects of titration as generally disclosed throughout FIGS. 1-15 in examples of the present disclosure may comprise (and/or may be implemented) in a manner complementary with and/or via at least some of substantially the same features and attributes as described in: (i) U.S. Pat. No. 8,938,299, issued on Jan. 20, 2015, and entitled “SYSTEM FOR TREATING SLEEP DISORDERED BREATHING”, and (ii) U.S. Patent Publication No. 2020/0147376, published on May 14, 2020, and entitled “MULTIPLE TYPE SLEEP APNEA”, each of which are hereby incorporated by reference in their entirety.

FIG. 11 is a block diagram schematically representing an example arrangement 3100 including patient's body 3102, including example target portions 3110-3134 at which at least some example sensing element(s) and/or stimulation elements may be employed to implement at least some examples of the present disclosure.

As shown in FIG. 11, patient's body 3102 comprises a head-and-neck portion 3110, including head 3112 and neck 3114. Head 3112 comprises cranial tissue, nerves, etc., and upper airway 3116 (e.g., nerves, muscles, tissues), etc. As further shown in FIG. 11, the patient's body 3102 comprises a torso 3120, which comprises various organs, muscles, nerves, other tissues, such as but not limited to those in pectoral region 3122 (e.g., lungs 3126, cardiac 3127), abdomen 3124, and/or pelvic region 3129 (e.g., urinary/bladder, anal, reproductive, etc.). As further shown in FIG. 11, the patient's body 3102 comprises limbs 3130, such as arms 3132 and legs 3134.

It will be understood that various sensing elements (and/or stimulation elements) as described throughout the various examples of the present disclosure may be deployed within the various regions of the patient's body 3102 to sense and/or otherwise diagnose, monitor, treat various physiologic conditions such as, but not limited to the above-described examples in association with FIGS. 1-10. In some such examples, a stimulation element 3117 (or at least a portion thereof) may be located in or near the upper airway 3116 for treating SDB (and/or near other nerves/muscles at the same or different location to treat SDB and/or other conditions) and/or a sensing element 3128 may be located anywhere within the neck 3114 and/or torso 3120 (or other body regions) to sense physiologic information for providing patient care (e.g., SDB, other).

In some examples, at least a portion of the stimulation element 3117 may comprise part of an implantable component/device, such as an IPG, whether full sized or sized as a microstimulator. The implantable components (e.g., IPG, other) may comprise a stimulation/control circuit, a power supply (e.g., non-rechargeable, rechargeable), communication elements, and/or other components. In some examples, the stimulation element 3117 also may comprise a stimulation electrode and/or stimulation lead connected to the IPG.

Further details regarding a location, structure, operation and/or use of the sensing element 3128, external element(s) 3150, and/or stimulation element 3117 are described above in association with at least FIGS. 1-10, and in particular, at least FIGS. 2B-5F and 9A-9C.

In some examples, at least a portion of the stimulation element 3117 may comprise part of an external component/device (e.g., external element 3150) such as, but not limited to, the external component comprising a PG (e.g., stimulation/control circuitry), power supply (e.g., rechargeable, non-rechargeable), and/other components. In some examples, a portion of the stimulation element 3117 may be implantable and a portion of the stimulation element 3117 may be external to the patient.

Accordingly, as further shown in FIG. 11, the various sensing element(s) 128 and/or stimulation element(s) 3117 implanted in the patient's body may be in wireless communication (e.g., connection 3137) with at least one external element 3150.

As further shown in FIG. 11, in some examples, the external element(s) 3150 may be implemented via a wide variety of formats such as, but not limited to, at least one of the formats 3151 including a patient support 3152 (e.g., bed, chair, sleep mat, other), wearable elements 3154 (e.g., finger, wrist, head, neck, shirt), noncontact elements 3156 (e.g., watch, camera, mobile device, other), and/or other elements 3158.

As further shown in FIG. 11, in some examples, the external element(s) 3150 may comprise one or more different modalities 3170 such as (but not limited to) a sensing portion 3171, stimulation portion 3172, power portion 3174, communication portion 3176, and/or other portion 3178. The different portions 3171, 3172, 3174, 3176, 3178 may be combined into a single physical structure (e.g., package, arrangement, assembly), may be implemented in multiple different physical structures, and/or with some of the different portions 3171, 3172, 3174, 3176, 3178 combined together in a single physical structure.

Among other such details, in some examples the external sensing portion 3171 and/or implanted sensing element 3128 may comprise an example implementation of, and/or at least some of substantially the same features and attributes as, the examples further described above in association with FIGS. 1-10, and in particular with regard to at least FIGS. 2B-5F and 9A-9C, respectively.

In some examples, the external stimulation portion 3172 and/or implanted stimulation element 3117 may comprise at least some of substantially the same features and attributes of at least the stimulation arrangements, as further described above in association with at least FIGS. 1, 2B-5F, 9A and/or other examples throughout the present disclosure.

In some examples, the external power portion 3174 and/or power components associated with implanted stimulation element 3117 may comprise at least some of substantially the same features and attributes of at least the stimulation electrode arrangements, as further described in association with at least FIGS. 1, 2B-5F, 9A and/or other examples throughout the present disclosure. In some such examples, the respective power portion, components, etc. may comprise a rechargeable power element (e.g., supply, battery, circuitry elements) and/or non-rechargeable power elements (e.g., battery). In some examples, the external power portion 3174 may comprise a power source by which a power component of the implanted stimulation element 3117 may be recharged.

In some examples, the wireless communication portion 3176 (e.g., connection/link at 3137) may be implemented via various forms of RF communication and/or other forms of wireless communication, such as (but not limited to) magnetic induction telemetry, BT, BT Low Energy (BLE), near infrared (NIF), near-field protocols, Wi-Fi, Ultra-Wideband (UWB), and/or other short range or long range wireless communication protocols suitable for use in communicating between implanted components and external components in a medical device environment.

Examples are not so limited as expressed by other portion 3178 via which other aspects of implementing medical care may be embodied in external element(s) 3150 to relate to the various implanted and/or external components described above.

FIGS. 12A-12D are diagrams including front and side views schematically representing patient anatomy and example methods relating to collapse patterns associated with upper airway patency. More specifically, FIGS. 12A-12D are a series of diagrams schematically representing at least some different upper airway collapse patterns, including an anterior-posterior (AP) collapse pattern (FIG. 12A), a concentric collapse pattern (FIG. 12B), a lateral collapse pattern (FIG. 12C), and an anterior-posterior (AP)—lateral collapse pattern (FIG. 12D). In addition to observing such collapse patterns and/or other collapse patterns, at least some aspects of such collapse patterns may be measured, such as via impedance sensing using implanted electrodes (e.g., sensing elements and/or stimulation elements), using externally applied arrays of electrodes, etc. such as described and illustrated in association with at least FIGS. 12A-12D. By determining an upper airway collapse pattern, some example arrangements may determine whether to apply stimulation via a hypoglossal nerve, via a glossopharyngeal nerve (and, optionally, specific fibers thereof), via an infrahyoid muscle-innervating nerve (including which single or multiple portions thereof to stimulate), via other non-hypoglossal nerve related to upper airway patency (e.g., glossopharyngeal nerve), and/or combinations of these nerves including unilateral and bilateral options.

At least some more specific details regarding FIGS. 12A-12D are further described below in relation to at least FIGS. 2A and 12E-12H.

FIG. 12E is a block diagram schematically representing an example sorting tool 1660 by which to sort and weigh a location, pattern, and degree of obstruction or patency. As shown in FIG. 12E, obstruction sorting tool 1660 includes functions for location detection 1662, pattern detection 1670, and degree detection 1680. In general terms, the location detection function 1662 operates to identify a site along the upper airway at which an obstruction occurs and which is believed to cause sleep disordered breathing. In one example, the location detection function 1662 includes a velum (soft palate) parameter 1664, an oropharynx-tongue base parameter 1666, and an epiglottis/larynx parameter 1668. Each respective parameter denotes an obstruction identified in the respective physiologic territories of the velum (soft palate), oropharynx-tongue base, and epiglottis which are generally illustrated for an example patient in FIG. 2A. In one aspect, these distinct physiologic territories define an array of vertical strata within the upper airway. Moreover, each separate physiologic territory (e.g., vertical portion along the upper airway) exhibits a distinct characteristic behavior regarding obstructions and associated impact on breathing during sleep. Accordingly, each physiologic territory responds differently to implantable upper airway stimulation.

With this in mind, the velum (soft palate parameter 1664 denotes obstructions taking place in the level of the region of the velum (soft palate), as illustrated in association with FIG. 2A. As previously described, FIG. 2A is a diagram including a side view schematically representing at least some anatomical features of the upper airway, as well as different sites or levels at which obstruction may occur. By determining a site or location of upper airway collapse, some example arrangements may determine whether to apply stimulation via a hypoglossal nerve, via an infrahyoid muscle-innervating nerve, via a glossopharyngeal nerve and/or selective fibers thereof, via other non-hypoglossal nerve related to upper airway patency, and/or combinations of these nerves and/or muscles including unilateral and bilateral options.

As shown in and referring back to FIG. 2A, a diagram 140 provides a side sectional view (cross hatching omitted for illustrative clarity) of a head-and-neck region 142 of a patient. In particular, an upper airway portion 150 extends from the mouth region 144 to a neck portion 155. The upper airway portion 150 includes a velum (soft palate) region 160, an oropharynx region 162, and an epiglottis region 164. The velum (soft palate) region 160 includes an area extending below sinus 161, and including the soft palate 160, approximately to the point at which tip 148 of the soft palate 146 meets a portion of tongue 147 at the back of the mouth region 144. The oropharynx region 162 extends approximately from the tip of the soft palate 146 (when in a closed position) along the base 152 of the tongue 147 until reaching approximately the tip region of the epiglottis 154. The epiglottis-larynx region 162 extends approximately from the tip of the epiglottis 1554 downwardly to a point above the esophagus 157.

As will be understood from FIG. 2A, each of these respective regions 160, 162, 164 within the upper airway correspond the respective velum parameter 164, oropharynx parameter 166, and epiglottis parameter 168, respectively, of FIG. 12E.

With further reference to FIG. 12E, in general terms the pattern detection function 1670 enables detecting and determining a particular pattern of an obstruction of the upper airway. In one example, the pattern detection function 1670 includes an antero-posterior parameter 1672, a lateral parameter 1674, a concentric parameter 1676, and composite parameter 1678.

The antero-posterior parameter 1672 of pattern detection function 1670 (FIG. 12E) denotes a collapse of the upper airway that occurs in the antero-posterior orientation, as further illustrated in the diagram 2510 of FIG. 12A. In FIG. 12A, arrows 2511 and 2512 indicate one example direction in which the tissue of the upper airway collapses, resulting in the narrowed air passage 2514. FIG. 12A is also illustrative of a collapse of the upper airway in the soft palate region (160 of FIG. 2A), whether or not the collapse occurs in an antero-posterior orientation. For example, in some instances, the velum (soft palate) region (160 of FIG. 2A) exhibits a concentric (e.g., circular) pattern of collapse, as shown in diagram 2520 of FIG. 12B.

The concentric parameter 1676 of pattern detection function 1670 (FIG. 12E) denotes a collapse of the upper airway that occurs in a concentric orientation, as further illustrated in the diagram 2520 of FIG. 12B. In FIG. 12B, arrows 2522 indicate the direction in which the tissue of the upper airway collapses, resulting in the narrowed air passage 2524.

The lateral parameter 1674 of pattern detection function 1670 (FIG. 12E) denotes a collapse of the upper airway that occurs in a lateral orientation, as further illustrated in the diagram 2530 of FIG. 12C. In FIG. 12C, arrows 2532 and 2533 indicate the direction in which the tissue of the upper airway collapses, resulting in the narrowed air passage 2535.

The composite parameter 1678 of pattern detection function 1670 (FIG. 12E) denotes a collapse of the upper airway portion that occurs via a combination of the other mechanisms (lateral, concentric, antero-posterior) or that is otherwise ill-defined from a geometric viewpoint but that results in a functional obstruction of the upper airway portion.

With further reference to obstruction sorting tool 1660 of FIG. 12E, in general terms the degree detection function or module 1680 indicates a relative degree of collapse or obstruction of the upper airway portion. In some examples, the degree detection function 1680 includes a none parameter 1682, a partial collapse parameter 1684, and a complete collapse parameter 1685. In some examples, the none parameter 1682 may correspond to a collapse of 25 percent or less, while the partial collapse parameter 1684 may correspond to a collapse of between about 25 to 75%, and the complete collapse parameter 1685 may correspond to a collapse of greater than 75 percent. In some examples, at least one respiration parameter may be sensed, such as using sensing portion 2000 of FIG. 9C, that includes respiratory obstruction information, such as neural activity which is indicative of a relative degree of collapse or obstruction of the upper airway.

It will be understood that various patterns of collapse occur at different levels of the upper airway portion and that the level of the upper airway in which a particular pattern of collapse appears may vary from patient-to-patient.

In some examples, obstruction sorting tool 1660 comprises a weighting function 1686 and score function 1687. In general terms, the weighting function 1686 assigns a weight to each of the location, pattern, and/or degree parameters (FIG. 12E) as one or more those respective parameters may contribute more heavily to the patient exhibiting sleep disordered breathing or to being more responsive to implantable upper airway stimulation. More particularly, each respective parameter (e.g., antero-posterior 1672, lateral 1674, concentric 1676, composite 1678) of each respective detection modules (e.g., pattern detection function 1670) is assigned a weight corresponding to whether or not the patient is eligible for receiving implantable upper airway stimulation. Accordingly, the presence of or lack of a particular pattern of obstruction (or location or degree) will be become part of an overall score (according to score parameter 1687) for an obstruction vector indicative how likely the patient will respond to therapy via an implantable upper airway stimulation system.

FIG. 12F is diagram (e.g., chart) 1690 schematically representing an index or scoring tool to sort and weigh a location, pattern, and degree of obstruction or patency for a particular patient. Chart 1690 combines information regarding location (1662 in FIG. 12E), pattern (1670 in FIG. 12E), and degree (1680 in FIG. 12E) into a single informational grid or tool by which the obstruction is documented for a particular patient and by which appropriate stimulation settings may be determined and applied according to the various examples of the present disclosure, such as but not limited to those in association with at least FIGS. 1-11, etc.

FIGS. 12G-12H are diagrams 1660A, 1690A like the diagrams 1660, 1690 of FIGS. 12E-12F, respectively, except with FIGS. 12G-12H further addressing an AP-lateral collapse pattern, which is depicted in diagram 2536 of FIG. 12D, provided as a parameter 1675 of a pattern detection function 1670 of FIG. 12G, and incorporated into the index of FIG. 12H.

As shown in FIG. 12D, this pattern comprises a combination of the anterior-posterior pattern (FIG. 12A) and the lateral pattern (FIG. 12C) with arrows 2537A, 2537B, 2537C indicating example directions in which the tissue of the upper airway collapses, resulting in the narrowed air passage 2538. The narrowed air passage 2538 may comprise a triangular shape in some examples. In some examples, the AP-lateral collapse pattern at a velum/soft palate (160 in FIG. 2A, 1664 in FIG. 12G) may respond better (e.g., increase patency) to stimulation of infrahyoid-based patency tissue (e.g., infrahyoid muscle-innervating nerve(s) and/or infrahyoid strap muscle(s)) than a concentric collapse pattern having a similar severity/completeness as the AP-lateral collapse pattern at the soft palate.

Accordingly, in some examples, the information sensed and collected via at least FIGS. 12E-12H may be used to determine whether to apply stimulation via a hypoglossal nerve, via a glossopharyngeal nerve, via an infrahyoid muscle-innervating nerve (including which single portion or multiple portions thereof to stimulate), via other non-hypoglossal nerves or muscles related to upper airway patency, and/or combinations of these nerves and/or muscles including unilateral and bilateral options. In some examples, other types of tissue may be stimulated, such as the phrenic nerve and/or diaphragm.

FIG. 13 schematically represents an example care engine 2400 by which at least some of substantially the same features and attributes of the examples of FIGS. 11-12H may be implemented in association with control portion 2500 (FIG. 14). In some examples, care engine 2400 may comprise at least some of substantially the same features and/or attributes as care engine 1311 of FIG. 6A.

FIG. 14 schematically represents an example control portion 2500 by which at least some of substantially the same features and attributes of the examples of FIGS. 11-12H may be implemented in association with control portion care engine 2400 (FIG. 13). In some examples, control portion 2500 may comprise at least some of substantially the same features and/or attributes as control portion 1300 of FIG. 6A, control portion 1328 of FIG. 6B, and/or control portion 916 of FIG. 9B.

FIG. 15 schematically represents an example user interface 2540 by which at least some of substantially the same features and attributes of the examples of FIGS. 11-12H may be implemented in association with control portion 2500 (FIG. 14) and/or care engine 2400 (FIG. 13). In some examples, user interface 2540 may comprise at least some of substantially the same features and/or attributes as user interface 1420 of FIG. 7.

In accordance with the examples described above, at least one fiber of the glossopharyngeal nerve may be selectively stimulated to promote upper airway patency. In some examples, the stimulation may be independent of detected SDB events and/or respiration information, such as when targeting stimulation of at least one afferent fiber and/or at least one efferent fiber to activate at least one pharyngeal constrictor muscles. In some examples, the stimulation may be timed using respiration information. In some examples, the stimulation may be selectively applied, such as applying to different target tissue at different times and/or simultaneously for treating SDB.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein.

1-52. (canceled)

53. A method, comprising:
selectively stimulating, via at least one stimulation element, at least one fiber of a glossopharyngeal nerve of a patient to promote upper airway patency.

54. The method of claim 53, wherein selectively stimulating comprises stimulating the at least one fiber while not stimulating other fibers at a target location of the glossopharyngeal nerve.

55. The method of claim 53, wherein selectively stimulating comprises stimulating the at least one fiber at a target location of the glossopharyngeal nerve to stimulate efferent fibers of the glossopharyngeal nerve while not stimulating afferent fibers of the glossopharyngeal nerve.

56. The method of claim 53, wherein selectively stimulating comprises stimulating the at least one fiber at a target location of the glossopharyngeal nerve to stimulate select afferent fibers of the glossopharyngeal nerve while not stimulating efferent fibers and other afferent fibers of the glossopharyngeal nerve.

57. The method of claim 53, wherein selectively stimulating comprises stimulating at least one of:
efferent fibers and at least one afferent fiber of the glossopharyngeal nerve, while not stimulating other afferent fibers of the glossopharyngeal nerve; and
at least one efferent fiber of the glossopharyngeal nerve as timed with respiration information and at least one afferent fiber of the glossopharyngeal nerve independent of the respiration information and stimulated using stimulation energy level below a threshold.

58. The method of claim 53, wherein the at least one fiber comprises an afferent fiber of the glossopharyngeal nerve and selectively stimulating the afferent fiber of the glossopharyngeal nerve further comprises inducing a physiologic response and thereby causing at least one of maintaining and increasing upper airway patency.

59. The method of claim 58, wherein the physiologic response is associated with a reflex opening activity associated with upper airway patency.

60. The method of claim 58, wherein the physiologic response comprises at least one of:
recruiting mechanoreceptors; and
recruiting chemoreceptors.

61. The method of claim 60, wherein the physiologic response causes at least one of:
reflex opening of the upper airway; and
priming of a sensory pathway associated with upper airway patency and reflex opening of the upper airway.

62. The method of claim 53, wherein the at least one fiber comprises an efferent fiber of the glossopharyngeal nerve and selectively stimulating the at least one fiber of the glossopharyngeal nerve comprises selectively activating upper airway patency-related muscle selected from a group consisting of:
at least one stylopharyngeus muscle;
at least one pharyngeal constrictor muscle; and
a combination thereof.

63. A method, comprising:
stimulating, via at least one stimulation element, at least one glossopharyngeal-related tissue of a patient to promote upper airway patency.

64. The method of claim 63, wherein the at least one glossopharyngeal-related tissue is selected from:
at least one stylopharyngeus muscle; and
at least one fiber of a glossopharyngeal nerve.

65. The method of claim 64, wherein the at least one glossopharyngeal-related tissue comprises at least one stylopharyngeus muscle and at least one fiber of the glossopharyngeal nerve.

66. The method of claim 63, wherein stimulating comprises selectively stimulating at least one fiber of a glossopharyngeal nerve.

67. The method of claim 63, wherein stimulating comprises selectively stimulating at least one fiber of a glossopharyngeal nerve while not stimulating other fibers at a target location of the glossopharyngeal nerve.

68. The method of claim 63, wherein stimulating causes, without activating at least one of coughing and trachea closure, at least one of:
activating the stylopharyngeus muscle without activating at least one of coughing and trachea closure;
activating at least one pharyngeal constrictor muscle without activating at least one of coughing and trachea closure; and
priming of a sensory pathway associated with upper airway patency and reflex opening of the upper airway.

69. A device, comprising:
a control portion configured to stimulate, via at least one stimulation element, at least one glossopharyngeal-related tissue.

70. The device of claim 69, wherein the at least one glossopharyngeal-related tissue comprises at least one stylopharyngeus muscle and at least one fiber of a glossopharyngeal nerve.

71. The device of claim 69, wherein the control portion is configured to selectively stimulate at least one fiber of a glossopharyngeal nerve while not stimulating other fibers at a target location of the glossopharyngeal nerve.

72. The device of claim 69, wherein the control portion is configured to selectively stimulate the at least one glossopharyngeal-related tissue without activating at least one of coughing and trachea closure.
